SEQUENCE LISTING (92 sequences)

SEQ ID NO: 1 - DNA, 15 bp, synthetic construct
  source 1..15: mol_type = other DNA
cgggccacggcgatt

SEQ ID NO: 2 - DNA, 23 bp, synthetic construct
  source 1..23: mol_type = other DNA
ggagtgggtagggtgtgtcataa

SEQ ID NO: 3 - DNA, 22 bp, synthetic construct
  source 1..22: mol_type = other DNA
ctacgagaccaagtgcaatccc

SEQ ID NO: 4 - DNA, 21 bp, synthetic construct
  source 1..21: mol_type = other DNA
aatcgccagccaattctcttt

SEQ ID NO: 5 - DNA, 20 bp, synthetic construct
  source 1..20: mol_type = other DNA
agctgctcacaaccgattca

SEQ ID NO: 6 - DNA, 20 bp, synthetic construct
  source 1..20: mol_type = other DNA
gtacattcttgccgcgcatc

SEQ ID NO: 7 - DNA, 20 bp, synthetic construct
  source 1..20: mol_type = other DNA
gagggctggcaacttagagg

SEQ ID NO: 8 - DNA, 21 bp, synthetic construct
  source 1..21: mol_type = other DNA
acaagctttgagtgcaagaga

SEQ ID NO: 9 - DNA, 18 bp, synthetic construct
  source 1..18: mol_type = other DNA
ccccctggctcactacga

SEQ ID NO: 10 - DNA, 20 bp, synthetic construct
  source 1..20: mol_type = other DNA
cactgaagccaccagcatgt

SEQ ID NO: 11 - DNA, 20 bp, synthetic construct
  source 1..20: mol_type = other DNA
ccgaccatatctgccaggac

SEQ ID NO: 12 - DNA, 20 bp, synthetic construct
  source 1..20: mol_type = other DNA
gcccttcagggtgaaatcca

SEQ ID NO: 13 - DNA, 22 bp, synthetic construct
  source 1..22: mol_type = other DNA
gccagaaccagctctataacga

SEQ ID NO: 14 - DNA, 19 bp, synthetic construct
  source 1..19: mol_type = other DNA
ggccacgtctcttgtccaa

SEQ ID NO: 15 - DNA, 19 bp, synthetic construct
  source 1..19: mol_type = other DNA
ctgccgctttgcaggtgta

SEQ ID NO: 16 - DNA, 21 bp, synthetic construct
  source 1..21: mol_type = other DNA
cattgtgggcaaggtgctatt

SEQ ID NO: 17 - DNA, 23 bp, synthetic construct
  source 1..23: mol_type = other DNA
aaactacaggtcaagtggtagcc

SEQ ID NO: 18 - DNA, 21 bp, synthetic construct
  source 1..21: mol_type = other DNA
tcctgcataagcacatcctga

SEQ ID NO: 19 - DNA, 20 bp, synthetic construct
  source 1..20: mol_type = other DNA
tgagccgcattctgaacgag

SEQ ID NO: 20 - DNA, 20 bp, synthetic construct
  source 1..20: mol_type = other DNA
gatgactgcgatccagagga

SEQ ID NO: 21 - DNA, 20 bp, synthetic construct
  source 1..20: mol_type = other DNA
ggacgacacccagcgtttat

SEQ ID NO: 22 - DNA, 20 bp, synthetic construct
  source 1..20: mol_type = other DNA
cgctcccatagttcaccgtg

SEQ ID NO: 23 - DNA, 21 bp, synthetic construct
  source 1..21: mol_type = other DNA
ccaaggttgatgcactgatgg

SEQ ID NO: 24 - DNA, 20 bp, synthetic construct
  source 1..20: mol_type = other DNA
tgtcagagacatgcgtctgc

SEQ ID NO: 25 - DNA, 23 bp, synthetic construct
  source 1..23: mol_type = other DNA
catctacacagtttgatgctgct

SEQ ID NO: 26 - DNA, 21 bp, synthetic construct
  source 1..21: mol_type = other DNA
gcagttttgtcagttcaggga

SEQ ID NO: 27 - DNA, 18 bp, synthetic construct
  source 1..18: mol_type = other DNA
cacgaagcaggccaatgg

SEQ ID NO: 28 - DNA, 20 bp, synthetic construct
  source 1..20: mol_type = other DNA
ctctgccctcgtgggtttgt

SEQ ID NO: 29 - DNA, 20 bp, synthetic construct
  source 1..20: mol_type = other DNA
gcaatactcgccttacggct

SEQ ID NO: 30 - DNA, 21 bp, synthetic construct
  source 1..21: mol_type = other DNA
tacacaccttggtagtacgcc

SEQ ID NO: 31 - DNA, 19 bp, synthetic construct
  source 1..19: mol_type = other DNA
tgcgagaaccccgagaagt

SEQ ID NO: 32 - DNA, 19 bp, synthetic construct
  source 1..19: mol_type = other DNA
gggaccagaacacctcgac

SEQ ID NO: 33 - DNA, 21 bp, synthetic construct
  source 1..21: mol_type = other DNA
attgggaattggcagaccaac

SEQ ID NO: 34 - DNA, 24 bp, synthetic construct
  source 1..24: mol_type = other DNA
ttgaagtatctaggatgccctgtg

SEQ ID NO: 35 - DNA, 19 bp, synthetic construct
  source 1..19: mol_type = other DNA
gcccctcattaagcccaag

SEQ ID NO: 36 - DNA, 21 bp, synthetic construct
  source 1..21: mol_type = other DNA
ttgtggtggtctgacagttcg

SEQ ID NO: 37 - DNA, 19 bp, synthetic construct
  source 1..19: mol_type = other DNA
ggaggaaaccaagcgccat

SEQ ID NO: 38 - DNA, 20 bp, synthetic construct
  source 1..20: mol_type = other DNA
cttgacggcgtggtagatgt

SEQ ID NO: 39 - DNA, 23 bp, synthetic construct
  source 1..23: mol_type = other DNA
tgtcttggaatgcactgtatctc

SEQ ID NO: 40 - DNA, 22 bp, synthetic construct
  source 1..22: mol_type = other DNA
cccagtaaggctgtaaatgctc

SEQ ID NO: 41 - DNA, 20 bp, synthetic construct
  source 1..20: mol_type = other DNA
ctacagtacagccaggcagg

SEQ ID NO: 42 - DNA, 20 bp, synthetic construct
  source 1..20: mol_type = other DNA
gaactggaagacaaggccca

SEQ ID NO: 43 - DNA, 24 bp, synthetic construct
  source 1..24: mol_type = other DNA
gcattcctgtggcatgtatgactt

SEQ ID NO: 44 - DNA, 20 bp, synthetic construct
  source 1..20: mol_type = other DNA
cccccagcaactccagattt

SEQ ID NO: 45 - DNA, 20 bp, synthetic construct
  source 1..20: mol_type = other DNA
ccagtttgggcatcaggaga

SEQ ID NO: 46 - DNA, 20 bp, synthetic construct
  source 1..20: mol_type = other DNA
agcatgaagttgggctcgaa

SEQ ID NO: 47 - DNA, 20 bp, synthetic construct
  source 1..20: mol_type = other DNA
gacttcacccagctcaacga

SEQ ID NO: 48 - DNA, 20 bp, synthetic construct
  source 1..20: mol_type = other DNA
atgctcgttctgcccattca

SEQ ID NO: 49 - DNA, 20 bp, synthetic construct
  source 1..20: mol_type = other DNA
cagtgaggcaagaccgaagt

SEQ ID NO: 50 - DNA, 21 bp, synthetic construct
  source 1..21: mol_type = other DNA
cggtgtccaaacttggtgaag

SEQ ID NO: 51 - DNA, 23 bp, synthetic construct
  source 1..23: mol_type = other DNA
ttccagaataccaacgtcaaagg

SEQ ID NO: 52 - DNA, 21 bp, synthetic construct
  source 1..21: mol_type = other DNA
gttttcggccagcagctattg

SEQ ID NO: 53 - DNA, 20 bp, synthetic construct
  source 1..20: mol_type = other DNA
atctccgactacgcccagac

SEQ ID NO: 54 - DNA, 19 bp, synthetic construct
  source 1..19: mol_type = other DNA
cgtgacgacccgtttcagg

SEQ ID NO: 55 - DNA, 21 bp, synthetic construct
  source 1..21: mol_type = other DNA
cggctgacgtgtgcagtaata

SEQ ID NO: 56 - DNA, 17 bp, synthetic construct
  source 1..17: mol_type = other DNA
ggcgccggaaagctgta

SEQ ID NO: 57 - DNA, 20 bp, synthetic construct
  source 1..20: mol_type = other DNA
cccagctcaaacaacacagc

SEQ ID NO: 58 - DNA, 20 bp, synthetic construct
  source 1..20: mol_type = other DNA
aggtagcatgccaggagaga

SEQ ID NO: 59 - DNA, 20 bp, synthetic construct
  source 1..20: mol_type = other DNA
atccgttacaagcgtcaggg

SEQ ID NO: 60 - DNA, 20 bp, synthetic construct
  source 1..20: mol_type = other DNA
tccccgtagtctgtgaggtc

SEQ ID NO: 61 - DNA, 20 bp, synthetic construct
  source 1..20: mol_type = other DNA
cgccttttactcgttgctca

SEQ ID NO: 62 - DNA, 20 bp, synthetic construct
  source 1..20: mol_type = other DNA
ctgtcaacagtgaggggacc

SEQ ID NO: 63 - DNA, 20 bp, synthetic construct
  source 1..20: mol_type = other DNA
ccaggtcccctggtgaaatc

SEQ ID NO: 64 - DNA, 20 bp, synthetic construct
  source 1..20: mol_type = other DNA
cgctgttggctgttctgttc

SEQ ID NO: 65 - DNA, 20 bp, synthetic construct
  source 1..20: mol_type = other DNA
agggagctcacagcgtgtat

SEQ ID NO: 66 - DNA, 20 bp, synthetic construct
  source 1..20: mol_type = other DNA
aggtcttggctgcagttctc

SEQ ID NO: 67 - DNA, 22 bp, synthetic construct
  source 1..22: mol_type = other DNA
catggacacaggataatggctg

SEQ ID NO: 68 - DNA, 23 bp, synthetic construct
  source 1..23: mol_type = other DNA
aggggttggttgctcaataaaaa

SEQ ID NO: 69 - DNA, 22 bp, synthetic construct
  source 1..22: mol_type = other DNA
tgccaattatgtttgccagttt

SEQ ID NO: 70 - DNA, 23 bp, synthetic construct
  source 1..23: mol_type = other DNA
tccattagggttttcagcacatt

SEQ ID NO: 71 - DNA, 22 bp, synthetic construct
  source 1..22: mol_type = other DNA
cattcctttggtatcagacccg

SEQ ID NO: 72 - DNA, 21 bp, synthetic construct
  source 1..21: mol_type = other DNA
ccctgaacgagatgccttcat

SEQ ID NO: 73 - DNA, 19 bp, synthetic construct
  source 1..19: mol_type = other DNA
ggccagttcagtgctgtcc

SEQ ID NO: 74 - DNA, 21 bp, synthetic construct
  source 1..21: mol_type = other DNA
gctcctgtcaccacatagtca

SEQ ID NO: 75 - DNA, 20 bp, synthetic construct
  source 1..20: mol_type = other DNA
cggcgtctctccttccttgt

SEQ ID NO: 76 - DNA, 21 bp, synthetic construct
  source 1..21: mol_type = other DNA
tactcaagggcagcgcttagc

SEQ ID NO: 77 - DNA, 20 bp, synthetic construct
  source 1..20: mol_type = other DNA
tcctgggagcaggttgtttg

SEQ ID NO: 78 - DNA, 20 bp, synthetic construct
  source 1..20: mol_type = other DNA
agaccttcagaagccccaga

SEQ ID NO: 79 - DNA, 20 bp, synthetic construct
  source 1..20: mol_type = other DNA
tgctcacttccccatcaacg

SEQ ID NO: 80 - DNA, 19 bp, synthetic construct
  source 1..19: mol_type = other DNA
gcttgctgaatcaaagccg

SEQ ID NO: 81 - DNA, 22 bp, synthetic construct
  source 1..22: mol_type = other DNA
tgcggagtctgtggtgtaccta

SEQ ID NO: 82 - DNA, 18 bp, synthetic construct
  source 1..18: mol_type = other DNA
gattcaccgccaccttgg

SEQ ID NO: 83 - DNA, 20 bp, synthetic construct
  source 1..20: mol_type = other DNA
gcagtcttccagtgtggtgt

SEQ ID NO: 84 - DNA, 20 bp, synthetic construct
  source 1..20: mol_type = other DNA
gatagagccgttgatccggg

SEQ ID NO: 85 - DNA, 20 bp, synthetic construct
  source 1..20: mol_type = other DNA
aaccagcgcatggacagtta

SEQ ID NO: 86 - DNA, 20 bp, synthetic construct
  source 1..20: mol_type = other DNA
gacttgaccaccgaacccat

SEQ ID NO: 87 - DNA, 19 bp, synthetic construct
  source 1..19: mol_type = other DNA
gctcttcaacgccatcaaa

SEQ ID NO: 88 - DNA, 18 bp, synthetic construct
  source 1..18: mol_type = other DNA
caggggcactcgattctg

SEQ ID NO: 89 - DNA, 20 bp, synthetic construct
  source 1..20: mol_type = other DNA
ggaggcatccacctacgatg

SEQ ID NO: 90 - DNA, 20 bp, synthetic construct
  source 1..20: mol_type = other DNA
gggagaagatggcgggtatg

SEQ ID NO: 91 - DNA, 22 bp, synthetic construct
  source 1..22: mol_type = other DNA
ctgcttgatggcattacgagaa

SEQ ID NO: 92 - DNA, 20 bp, synthetic construct
  source 1..20: mol_type = other DNA
tgcaggctttgcagaaccta

(19) United States
(12) Patent Application Publication
(10) Pub. No.: US 2026/0085356 A1
(43) Pub. Date: Mar. 26, 2026

(54) BIOMARKER FOR DIAGNOSING PRE-CHEMOTHERAPY RESISTANCE IN SOLID CANCER PATIENTS AND METHOD FOR PROVIDING INFORMATION FOR DIAGNOSING PRE-CHEMOTHERAPY RESISTANCE, USING SAME

(71) Applicants: THE ASAN FOUNDATION, Seoul (KR); UNIVERSITY OF ULSAN FOUNDATION FOR INDUSTRY COOPERATION, Ulsan (KR)

(72) Inventors: Dong-Myung SHIN, Seoul (KR); Yong-Mee CHO, Seoul (KR); Yonghwan KIM, Seoul (KR)

(73) Assignees: THE ASAN FOUNDATION, Seoul (KR); UNIVERSITY OF ULSAN FOUNDATION FOR INDUSTRY COOPERATION, Ulsan (KR)

(21) Appl. No.: 19/109,932

(22) PCT Filed: Sep. 7, 2023
(86) PCT No.: PCT/KR2023/013456

§ 371 Date: Mar. 7, 2025

(30) Foreign Application Priority Data
Sep. 7, 2022 (KR) 10-2022-0113349

(51) Int. Cl. C12Q 1/6886; A61K 33/243; C12Q 1/6809

(52) U.S. Cl. CPC C12Q 1/6886; A61K 33/243; C12Q 1/6809; C12Q 2600/106; C12Q 2600/158

(57) ABSTRACT

Biomarkers useful for detecting resistance to platinum-based chemotherapeutic agents (e.g., cisplatin) administered as pre-chemotherapy (NAC) for solid tumors (e.g., muscle-invasive bladder cancer (MIBC)). The biomarkers include glutathione-related genes, the expression levels of which can be used to determine the NAC resistance. The biomarkers are used in a method of determining chemotherapy resistance and in a method of treating a cancer in a subject.

### TECHNICAL FIELD

The present invention identifies that there is a connection between resistance to neoadjuvant chemotherapy in patients with solid cancer and the glutathione pathway, and provides a glutathione metabolite as a biomarker for diagnosing resistance to neoadjuvant chemotherapy in patients with solid cancer. Using the biomarker composition, kit, and information providing method of the present invention, it is possible to easily diagnose whether a solid cancer patient exhibits resistance to neoadjuvant chemotherapy.

The present invention is derived from research conducted as part of the individual basic research project of the Korea Ministry of Science and ICT [Assignment Unique Number 1711192152 Detailed Assignment Number 2021R1A2C2005790].

### BACKGROUND

Bladder cancer (BC) has a high incidence and mortality rate, and is estimated to be the 6th most common malignancy in men worldwide, with an estimated 573,278 new cases and 212,536 deaths in 2020. Bladder cancer exhibits considerable clinical and pathologic heterogeneity. Depending on the extent of bladder wall invasion, BC is classified as non-muscle invasive BC (NMIBC; Ta, intraepithelial carcinoma, T1) or muscle invasive BC (MIBC; ≥T2). NMIBC accounts for most BC patients (70-80%) at initial diagnosis. NMIBC is usually a non-aggressive tumor and is managed with transurethral resection of bladder tumor (TURBT) followed by intravesical chemotherapy or BCG vaccination. However, because MIBC has a high recurrence and progression rate, lifelong surveillance costs are high. MIBC, which accounts for 25% of tumor incidence, can rapidly progress to metastatic BC and is responsible for most patient mortality, and appropriate management of MIBC is important to reduce BC mortality. After diagnosis via TURBT, radical cystectomy with preoperative cisplatin-based chemotherapy, i.e. neoadjuvant chemotherapy (NAC), is currently the standard for MIBC treatment. NAC is effective in only 30-40% of cases, and the remaining nonresponsive patients may experience drug toxicity without oncological benefit and may delay definitive treatment. However, the current clinicopathologic features of MIBC patients are inadequate to prospectively predict NAC response. There is no established treatment strategy for nonresponsive patients. Therefore, identifying the under lying mechanisms of chemotherapy resistance and predictive biomarkers to improve the accuracy and efficacy of clinical management of MIBC may contribute to a significant unmet clinical need.

Genome-wide gene expression profiling has generated precise molecular insights into the complexity of the heterogeneous behavior of MIBC, including prognostic assessment and treatment response. For example, patients with the p53-like molecular subtype respond poorly to NAC. In contrast, patients with basal-like tumors exhibited the greatest oncological benefit, with increased overall survival (OS) after NAC compared to an unmatched cohort of patients who underwent solo surgery. However, previous studies have reported a decreased NAC response rate in patients with the basal squamous (Ba/Sq) expression subtype. In addition, a study integrating previously published primary data sets observed no statistically significant association in predicting NAC response via consensus classification. These conflicting results highlight the complexity of determining response to NAC in MIBC. Therefore, identifying the underlying mechanisms of chemotherapy resistance and predictive biomarkers to improve the accuracy and efficacy of clinical management of MIBC contributes to a significant unmet clinical need.

To determine the biological basis for molecular heterogeneity in MIBC NAC response, the present inventors performed gene expression profiling of MIBC patient tumors in four independent cohorts. It defines the importance of coordinated upregulation of metabolic pathways to control the levels and activity of glutathione (GSH), which is a highly abundant nonprotein thiol tripeptide composed of glutamate, cysteine, and glycine. Furthermore, by integrating digitalized analysis of MIBC tumor tissue immunostaining using a machine learning-based tumor-stroma classifier, live cell real-time GSH monitoring, in vitro cell culture, and in vivo animal model analyses, the present inventors demonstrated the biological significance and clinical relevance of GSH dynamics as a potential predictive biomarker for MIBC NAC response and potentially as a novel therapeutic target to resensitize chemoresistant MIBC.

### DISCLOSURE

### Technical Problem

The present invention has been devised to solve the above problems and meet the above needs, and the present invention aims to identify a mechanism associated with resistance to neoadjuvant chemotherapy in a solid cancer patient and to provide this as a method for diagnosing the occurrence of resistance.

The present invention aims to provide a biomarker composition capable of diagnosing the occurrence of resistance to neoadjuvant chemotherapy in a solid cancer patient.

The present invention also aims to provide a kit capable of diagnosing the occurrence of resistance to neoadjuvant chemotherapy in a solid cancer patient.

### Technical Solution

To solve the above-mentioned problems, the present invention provides a biomarker composition for diagnosing chemotherapy resistance and predicting prognosis in a solid cancer patient, comprising a glutathione metabolite or a combination thereof.

In the present invention, the solid cancer includes, but is not limited to, bladder cancer, colon cancer, stomach cancer, lung cancer, lung adenocarcinoma, breast invasive ductal carcinoma, colon adenocarcinoma, prostate adenocarcinoma, bladder urothelial carcinoma, lung squamous cell carcinoma, cutaneous melanoma, cancer of unknown primary, pancreatic adenocarcinoma, glioblastoma multiforme, colorectal adenocarcinoma, high grade serous ovarian cancer, stomach adenocarcinoma, renal clear cell carcinoma, esophageal adenocarcinoma, testicular cancer, and intrahepatic cholangiocarcinoma, and is most preferably muscle-invasive bladder cancer (hereinafter also referred to as MIBC).

In the present invention, the glutathione metabolite includes GLS1, PSAT1, CBS, GCLC, GCLM, Gluta (GSR), GlnRS (QARS1), GGT7, Perox (PRDX1), PLOD2, RPAP1, RPL9, MITF, CD44v6, CDK1, FZD9, GAD2, PPP2R5A, non-p (b-catenin), B-catenin, SALL4, SOX2, TFCP2L1, TFEB, ICAM1, TRAF2, TRAF6, IL15RA, AFAP1, CARD16, CD11c, CD73, CD3Z, EB13 (IL27B), EMX1, DNMT3L, DPII2, EGR2, FYB1, GADD45B, HCFC1R1, KCTD14, MTCH1, OCT2, PCDHB9, PPIL2, RFX7, SLC15A3, TNFAIP8, ANPEP, BDN, EOGT, FOXA1, KIFC2, KIR3DL1, NOTUM, USP2, CCDC6, CK5 (KRT5), CK20 (KRT20), GATA3, BHLHE40, C11orf95, REX7, COX17, NTSE, ITGAX, KIAA1671, NT5E, POU2F2, PLEK, POSTN, PPP1R35, THBS2, and INFAIP8.

In the present invention, the chemotherapy resistance may mean resistance to a platinum agent (e.g., cisplatin), and the platinum agent may be administered as neoadjuvant chemotherapy. That is, the present invention provides glutathione metabolites as biomarkers for diagnosing resistance to platinum agents treated with neoadjuvant (anticancer) chemotherapy.

The present invention also provides a composition for diagnosing chemotherapy resistance in a muscle-invasive bladder cancer patient, comprising an agent for measuring the expression of glutathione metabolite. In the present invention, the agent for measuring the expression of glutathione metabolite may include a peptide, antibody, and primer that specifically bind to a gene of the metabolite.

The present invention also provides a kit for diagnosing chemotherapy resistance in a muscle-invasive bladder cancer patient, comprising the composition for diagnosing chemotherapy resistance in a muscle-invasive bladder cancer patient.

According to an aspect, the present invention relates to a method for providing information for diagnosing chemotherapy resistance in a muscle-invasive bladder cancer patient, the method including a) measuring the expression level of glutathione metabolite from a sample isolated from a muscle-invasive bladder cancer patient; b) comparing the expression level of the glutathione metabolite with that of a normal control group sample; and c) determining that there is chemotherapy resistance when the expression level of glutathione metabolite measured from the sample isolated from the muscle-invasive bladder cancer patient is different from the level of the control group sample.

In the present invention, the expression level of the glutathione metabolite may be measured by Western blot, enzyme-linked immunosorbent assay (ELISA), immunohistochemical staining, immunoprecipitation, immunofluorescence, transcriptome, epigenome, or quantitative real-time PCR techniques.

In the present invention, saliva, urine, tissue, whole blood, serum or plasma may be used as a sample for analyzing the expression of the glutathione metabolite, but is not limited thereto.

The present invention also relates to a method for providing information for determining a treatment method for a muscle-invasive bladder cancer patient, the method including a) measuring the expression level of glutathione metabolite from a sample isolated from a muscle-invasive bladder cancer patient; b) comparing the expression level of the glutathione metabolite with that of a normal control group sample; and c) determining that there is chemotherapy resistance when the expression level of glutathione metabolite measured from the sample isolated from the muscle-invasive bladder cancer patient is different from the level of the control group sample.

### Advantageous Effect

The present invention identifies that the glutathione pathway is associated with resistance to neoadjuvant chemotherapy in a solid cancer patient, and provides a biomarker composition capable of diagnosing the occurrence of resistance to neoadjuvant chemotherapy in a solid cancer patient.

The present invention also provides a combination of biomarkers capable of diagnosing the occurrence of resistance to neoadjuvant chemotherapy in a solid cancer patient.

When the biomarker composition of the present invention is used, it is possible to non-invasively, simply, and quickly diagnose whether resistance to neoadjuvant chemotherapy has developed.

### BRIEF DESCRIPTION OF THE DRAWINGS

FIGS. 1A to 1E are schematic diagrams showing the procedures of immunostaining digital analysis using machine learning-based tumor-stroma classifier, live cell monitoring, in vitro cell culture, and in vivo animal model experimental, by integrating multi-cohort transcriptome and clinical data analysis of patients with muscle-invasive bladder cancer (MIBC). Referring to FIG. 1, the present invention identifies the biological significance and clinical relevance of glutathione dynamics as a potential predictive biomarker for MIBC response to preoperative cisplatin-based neoadjuvant chemotherapy and potentially as a novel therapeutic target to resensitize chemoresistant MIBC.

FIGS. 2A to 2G show transcriptome profiling based on neoadjuvant chemotherapy response of muscle-invasive bladder cancer (MIBC). FIG. 2A is a schematic diagram of a multi-cohort analysis of MIBC patients with molecular features, NAC response, and clinical data. FIG. 2B shows a result of analyzing the principal components of the transcriptome of the AMC discovery cohort after classifying MIBC patients into NAC response (R) and non-response (NR) groups (n=8/group, NR and R). FIG. 2C shows a heatmap analysis result of differentially expressed genes (DEGs) between the NR and R group transcriptomes. FIG. 2D shows a representative enrichment plot result using gene set enrichment analysis (GSEA) and gene sets associated with published molecular classifiers to stratify molecular MIBC subtypes (NES: normalized enrichment score; FDR: false discovery rate). FIGS. 2E and 2F are images showing the top 10 most enriched pathway maps (FIG. 2E) and a representative enriched gene network (FIG. 2F) identified through MetaCore analysis by comparing transcriptomes between the NAC NR and R groups of the present invention. Fold change values represent genes upregulated in red and downregulated in blue, overlaid on the gene network. FIG. 2G is a GSEA with representative enrichment plots of gene sets defining NAC response in the AMC discovery cohort. In FIG. 2G, the bubble plots are displayed in NES order through the size values according to −Log2 (FDR).

FIGS. 3A to 3I show results of analyzing the characteristics of NAC response molecules in different MIBC cohorts. FIG. 3A shows the top-scoring 10 gene sets (FIG. 3A left panel) and representative enrichment plots (FIG. 3A right panel) from GSEA analysis on transcriptome data sets of 39 patients (NR n=18, R n=21) in the AMC validation cohort. Here, gene sets are listed according to their normalized enrichment scores (NES). FIG. 3B shows representative images of TUBRT tumor specimens (×100 magnification, scale bar=100 μm) from the AMC validation cohort for assessing tumor-infiltrating lymphocytes (TILs). Quantitative data in FIG. 3B are presented as mean±SEM (*p<0.05, unpaired Student's t-tests). FIGS. 3C to 3E show results of MetaCore analysis comparing the transcriptomes between NAC NR and NAC NR, showing the R group results of the AMC validation cohort, which are the most enriched pathway map at that time (FIG. 3C), GO process (FIG. 3D), and representative enriched gene network related to detoxification of inorganic compounds (FIG. 3E). FIG. 3F shows the transcriptome data set for AMC validation and a description of the MIBC samples, as well as three independent external cohorts containing publicly available gene expression profiling data sets of pre-NAC TURBT specimens and records of pathologic response to NAC. In FIG. 3F, the number of MIBC samples used in each dataset and the significant gene sets identified in the GSEA analysis are shown in the “MIBC samples” and “Number of significant gene sets” columns, respectively. FIGS. 3G to 3I show GSEA analyses for three external cohorts using 30 gene sets differentially associated with NAC response in the discovery cohort. FIGS. 3G and 3H show the top-scoring significant gene sets in the “MDA MVAC” cohort (FIG. 3G) and representative enrichment plots of the GSH metabolism gene set in the “MDA DDMVAC” cohort (FIG. 3H) (ES: enrichment score; NOM p-value, nominal p-value; FDR: false discovery rate). FIG. 3I is a heatmap analysis result summarizing the FDR values from the GSEA analysis for AMC and three external validation cohorts.

FIGS. 4A to 4E show results of identifying biomarkers characterizing NAC response in the AMC discovery cohort. FIG. 4A is a result of a state-of-the-art analysis of the transcriptome of the AMC discovery cohort using the top 10 enriched gene sets (left) in the NAC NR group according to the present invention. FIG. 4A shows the richness of the gene sets (right image in FIG. 4A) and the heatmap of the corresponding target genes (bottom image in FIG. 4A). FIG. 4B shows a bubble plot of qPCR results for putative biomarker genes selected as significant genes according to state-of-the-art (GSEA) and gene network (MetaCore) analyses. FIG. 4c shows results of qPCR analysis to validate the expression of a subset of biomarker genes in the AMC cohort patients (NR n=17, R n=19). In FIG. 4C, expression is expressed as a percentage of human b2-microglobulin (B2M) expression. FIG. 4D shows the expression levels of proteins related to GSH and metabolic pathways (top image) or immune and cell adhesion processes (bottom image) in the AMC discovery cohort (n=8/group, NR and R). FIG. 4E shows representative images of the expression of genes related to GSH dynamics (GLS1, GSR, and GCLM) or CD11c immune response-related protein in NAC NR and R patients (×40 (top image) ×200 (bottom image) magnification, scale bar=100 mm). Quantitative results in this figure are presented as dot plots of mean±SEM (*p<0.05, **p<0.01, ***p<0.001, unpaired Student's t-test). The p-value and number of repetitions are specified in the source data set.

FIGS. 5A to 5E are images identifying the upregulation of GLS1 protein in NAC-resistant MIBC. FIG. 5A is a schematic workflow of digital pathology analysis using a machine learning approach to distinguish between tumor epithelial cells and stroma in IHC images according to the present invention. FIG. 5B is a bubble plot for digital pathology analysis of 11 proteins in tumor epithelial cells from the AMC validation cohort (NR n=19, R n=34). FIG. 5C shows the expression levels of GLS1, CD11c, and EOGT proteins in the tumor (top image) or stromal (bottom image) compartments of the AMC validation cohort. Quantitative results are presented as dot plots of mean±SEM (*p<0.05, unpaired Student's t-test). FIGS. 5D and 5E show the results of univariate and multivariate analyses of the expression of 41 putative proteins and clinicopathologic factors tested in the study based on the response to NAC. FIG. 5D is an image showing the correlation between expressions of GLS1 and six proteins that were identified as statistically significant based on high (red) or low (blue) expression of GLS1 in tumor epithelial cells (GLS1 high n=14, GLS1 low n=40). The average expression level of the indicated proteins in each group is represented by the p-value, and the exact p-value and number of repeated experiments are shown in the source dataset.

FIGS. 6AA to 6C show results of deep learning-based analysis on the transcriptome dataset of multi-cohort MIBC patients. FIGS. 6AA and 6AB are lists of molecular classifiers used to optimize deep learning-based analytical modeling for predicting NAC chemotherapy responsiveness in MIBC patient samples. FIG. 6B shows a main result of deep learning-based analyses performed using multiple patient cohorts and molecular classifiers. FIG. 6C is a figure showing the performance and validity of the BC p53-like pathway among the main result of deep learning-based analysis.

FIGS. 7A to 7D show results of identifying putative biomarkers for predicting NAC response in MIBC patients. FIG. 7A shows a result of logistic regression, random forest, and decision tree modeling in the deep learning-based multi-cohort transcriptome analysis modeling result. FIG. 7B shows a result of how two different molecular classifiers distinguish between NAC NR and R groups. FIG. 7C shows a result of a decision tree of important key genes within the molecular classifiers used in FIG. 7B.

FIG. 8 shows a result of deriving key genes important for predicting NAC responsiveness in MIBC patient samples and validating the differences in protein expression of these genes through digital pathology analysis.

FIGS. 9A to 9G show a process and result of discovering the optimal protein combination for predicting NAC responsiveness using a digital pathology dataset. FIG. 9A is an overview of the entire analysis process. FIG. 9B is a description of a recursive feature elimination analysis with cross-validation (RFECV) method used to select the most important variables from a specific dataset and optimize the performance of the model by eliminating unnecessary variables. FIG. 9C is a list of protein groups that can distinguish NAC responsiveness selected from the results of tumor compartments in a digital pathology dataset. FIG. 9D is a list of protein groups that can distinguish NAC responsiveness selected from the results of stroma compartments in a digital pathology dataset. FIG. 9E is a result of the optimal model derived through the results of FIGS. 9C and 9D. FIG. 9F shows a result of the optimal model of the tumor compartment derived above, expressed in terms of the importance of each variable using a decision tree model. FIG. 9G shows a result of the optimal model of the stroma compartment derived above, expressed in terms of the importance of each variable using a decision tree model.

FIG. 10 shows a validation result of the deep learning-based digital pathology model developed in this study.

### BEST MODES FOR CARRYING OUT THE INVENTION

Hereinafter, the present invention will be described in more detail. Advantages and features of the present invention, and a method of achieving them will be apparent with reference to the embodiments described below. However, the present invention is not limited to the embodiments disclosed below, and may be embodied in various forms. Rather, the description of the embodiments of the present invention is provided so that this disclosure will be thorough and complete and will fully convey the concept of the invention to those of ordinary skill in the art. Accordingly, the present invention is only defined by the scope of the appended claims. Like reference numerals designate like elements throughout the specification.

Unless otherwise defined, all terms (including technical and scientific terms) used in this specification will be used as meanings that can be commonly understood by those of ordinary skill in the art. In addition, terms defined in a commonly used dictionary are not interpreted ideally or excessively unless explicitly and specifically defined. The terms used in this specification are for the purpose of describing embodiments only and are not intended to limit the present invention. In this specification, the singular form also includes the plural form unless specifically specified in the phrase.

The present invention provides a biomarker composition for diagnosing chemotherapy resistance and predicting prognosis in a solid cancer patient, comprising a glutathione metabolite or a combination thereof. The present invention aims to identify that the glutathione pathway is associated with resistance to neoadjuvant chemotherapy in patients with solid cancer, and to diagnose resistance to neoadjuvant chemotherapy in patients with solid cancer based on the expression level of glutathione metabolites.

In the present invention, “metabolite” is an intermediate product or product of metabolism. These metabolites have diverse functions, including fuel, structure, signaling, stimulatory and inhibitory effects on enzymes, their own catalytic activity (usually as cofactors for enzymes), defense, and interactions with other organisms (e.g., pigments, aromatic compounds, pheromones). Primary metabolites are directly involved in normal growth, development, and reproduction, while secondary metabolites are not directly involved in these processes, but usually have important ecological functions.

Biological samples from which the above metabolites can be obtained include whole blood, plasma, serum, red blood cells, white blood cells (e.g., peripheral blood monocytes), ductal fluid, ascites, pleural efflux, nipple aspirate, lymph fluid (e.g., disseminated tumor cells in a lymph node), bone marrow aspirate, saliva, urine (pee), feces (i.e., excretions), sputum, bronchial lavage fluid, tears, fine needle aspirates, any other body fluid, tissue samples (e.g., tumor tissue), tumor biopsies (e.g., needle biopsics), lymph nodes (e.g., sentinel lymph node biopsics), surgical resections of tumors and cellular extracts thereof, preferably plasma. In addition, the metabolites may include substances produced by metabolism and metabolic processes, or substances resulting from chemical metabolic reactions by biological enzymes and molecules.

In the present invention, the glutathione metabolite includes GLS1, PSAT1, CBS, GCLC, GCLM, Gluta (GSR), GlnRS (QARS1), GGT7, Perox (PRDX1), PLOD2, RPAP1, RPL9, MITF, CD44v6, CDK1, FZD9, GAD2, PPP2R5A, non-p (b-catenin), B-catenin, SALL4, SOX2, TFCP2L1, TFEB, ICAM1, TRAF2, TRAF6, IL15RA, AFAP1, CARD16, CD11c, CD73, CD3Z, EBI3 (IL27B), EMX1, DNMT3L, DPH2, EGR2, FYB1, GADD45B, HCFC1R1, KCTD14, MTCH1, OCT2, PCDHB9, PPIL2, RFX7, SLC15A3, TNFAIP8, ANPEP, BDN, EOGT, FOXA1, KIFC2, KIR3DL1, NOTUM, USP2, CCDC6, CK5 (KRT5), CK20 (KRT20), GATA3, BHLHE40, C11orf95, REX7, COX17, NTSE, ITGAX, KIAA1671, NT5E, POU2F2, PLEK, POSTN, PPP1R35, TIIBS2, and INFAIP8.

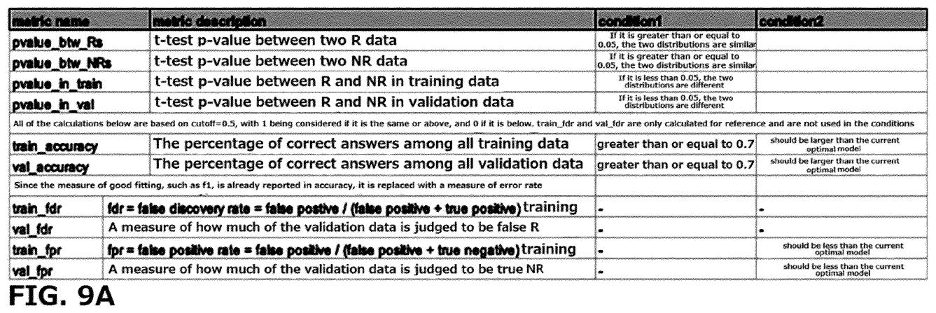

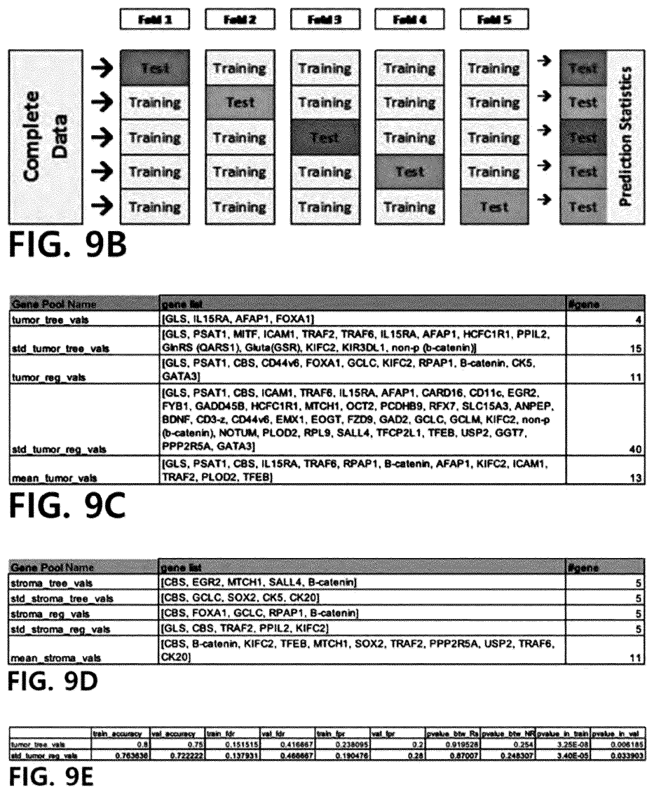

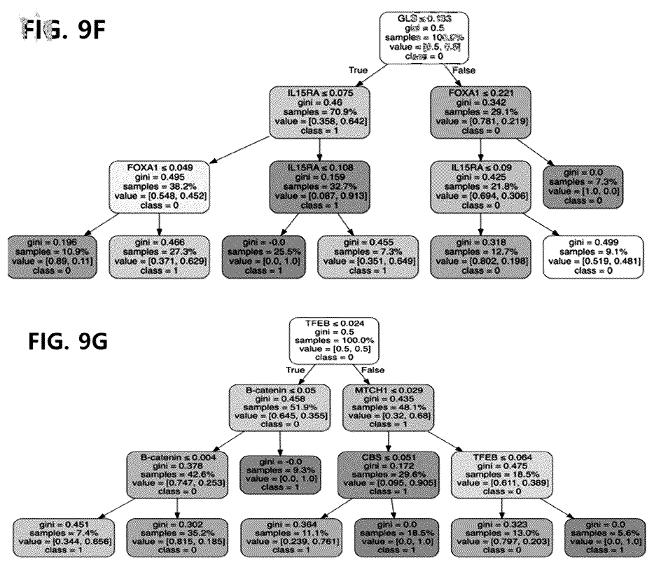

In the present invention, combinations of glutathione metabolites suitable for diagnosing neoadjuvant chemotherapy resistance in solid cancer patients include a first combination (GLS, IL 15RA, AFAP1, FOXA1), a second combination (GLS, PSAT1, MITF, ICAM1, TRAF2, TRAF6, IL 15RA, AFAP1, HCFC1R1, PPIL2, GinRS (QARS1), Gluta (GSR), KIFC2, KIR3DL 1, non-p (b-catenin)), a third combination (GLS, PSAT1, CBS, CD44v6, FOXA1, GCLC, KIFC2, RPAP1, B-catenin, CK5, GATA3), a fourth combination (GLS, PSAT1, CBS, ICAM1, TRAF6, IL15RA, AFAP1, CARD16, CD11c, EGR2, FYB1, GADD45B, HCFC1R1, MTCH1, OCT2, PCDHB9, RFX7, SLC15A3, ANPEP, BDNF, CD3-2, CD44v6, EMX1, EOGT, FZD9, GAD2, GCLC, GCLM, KIFC2, non-p (b-catenin), NOTUM, PLOD2, RPL9, SALLA, TFCP2L1, TFEB, USP2, GGT7, PPP2R5A, GATA3), a fifth combination (GLS, PSAT1, CBS, IL15RA, TRAF6, RPAP1, B-catenin, AFAP1, KIFC2, ICAM1, TRAF2, PLOD2, TFEB), a sixth combination (CBS, EGR2, MTCH1, SALL4, B-catenin), a seventh combination (CBS, GCLC, SOX2, CK5, CK20) an eighth combination (CBS, FOXA1, GCLC, RPAP1, B-catenin) a ninth combination (GLS, CBS, TRAF2, PPIL2, KIFC21, and a tenth combination (CBS, B-catenin, KIFC2, TFEB, MTCH1, SOX2, TRAF2, PPP2R5A, USP2, TRAF6, CK20) (see FIGS. 9C and 9D).

According to a preferred embodiment of the present invention, the expression levels of GLS1, SLC15A3, and TNFAIP8 may be higher in a patient group with resistance to neoadjuvant chemotherapy than in a control group without resistance to neoadjuvant chemotherapy in solid cancer patients.

According to a preferred embodiment of the present invention, the expression of EOGT, CD11c, KIFC2, CK5, CK20, CARD16, CD73, DNMT3L, FYB1, HCFC1R1, MTCH1, and PFX7 may decrease in a patient group with resistance to neoadjuvant chemotherapy than in a control group without resistance to neoadjuvant chemotherapy in solid cancer patients.

The metabolite biomarker of the present invention can be detected by a known method for measuring glutathione expression level and activity, such as Western blot, enzyme-linked immunosorbent assay (ELISA), immunohistochemical staining, immunoprecipitation, or immunofluorescence, and the expression level can be analyzed by analyzing it using a machine learning technique.

In the present invention, the biological sample can be pretreated to detect a metabolite biomarker. For example, the pretreatment may include filtration, distillation, extraction, separation, concentration, inactivation of interfering components, addition of reagents, etc.

In the present invention, a “quantitative device” means a device that provides quantitative numerical information on the presence or absence of a specific metabolite in a biological sample as well as its relative or absolute amount. Specifically, the quantitative device is a chromatograph, a mass spectroscopy (MS), or a nuclear magnetic resonance (NMR) spectrometer.

In the present invention, “chromatography” includes, but is not limited to, high performance liquid chromatography (HPLC), liquid-solid chromatography (LSC), paper chromatography (PC), thin-layer chromatography (TLC), gas-solid chromatography (GSC), liquid-liquid chromatography (LLC), foam chromatography (FC), emulsion chromatography (EC), gas-liquid chromatography (GLC), ion chromatography (IC), gel filtration chromatography (GFC), or gel permeation chromatography (GPC), and any quantitative chromatography commonly used in the art may be used.

In this specification, “mass spectrometry (MS)” means a process of measuring the mass of a target substance to analyze the chemical composition of the sample. Mass spectrometry produces charged molecules or molecular pieces through ionization of the target substance present in the sample, and provides information on mass by measuring the mass-to-charge ratio (m/z) and the abundance of gaseous ions. Such mass spectrometry devices include, but are not limited to, for example, MALDI-TOF (Matrix-Assisted Laser Desorption/Ionization Time of Flight), SELDI-TOF (Surface Enhanced Laser Desorption/Ionization Time of Flight), ESI-TOF (Electrospray ionization Time of Flight), LC-MS (liquid chromatography mass spectrometry) or LC-MS/MS (liquid chromatography-Mass Spectrometry/Mass Spectrometry).

The term “diagnosis” in the present invention includes determining the susceptibility of a subject to a particular disease or condition, determining whether a subject currently has a particular disease or disorder, determining the prognosis of a subject with a particular disease or disorder, or therametrics, e.g., monitoring the condition of a subject to provide information on the efficacy of a treatment.

In the present invention, the solid cancer includes, but is not limited to, bladder cancer, colon cancer, stomach cancer, lung cancer, lung adenocarcinoma, breast invasive ductal carcinoma, colon adenocarcinoma, prostate adenocarcinoma, bladder urothelial carcinoma, lung squamous cell carcinoma, cutaneous melanoma, cancer of unknown primary, pancreatic adenocarcinoma, glioblastoma multiforme, colorectal adenocarcinoma, high grade serous ovarian cancer, stomach adenocarcinoma, renal clear cell carcinoma, esophageal adenocarcinoma, testicular cancer, and intrahepatic cholangiocarcinoma.

In the present invention, the term “patient” may typically include not only humans but also other animals, such as other primates, rodents, dogs, cats, horses, sheep, pigs, etc. The ‘patient’ of the present invention includes subjects other than humans diagnosed with or suspected of having solid cancer.

In the present invention, the chemotherapy resistance means, but is not limited to, resistance to an anticancer agent used as neoadjuvant chemotherapy (particularly, a platinum agent (platinum complex compound-based anticancer agent)). In the present invention, the platinum agent includes heptaplatin, nedaplatin, boplatin, etc., and preferably means cisplatin.

The present invention also provides a use of an agent for measuring the expression of glutathione metabolites in diagnosing chemotherapy resistance in a solid cancer patient, and provides a method for diagnosing chemotherapy resistance in a solid cancer patient using the agent.

Hereinafter, in order to help understanding of the present invention, examples will be described in detail.

### Example 1

### Experimental Method

### 1-1. Data and Code Validation

All raw and processed transcriptome data from the Seoul Asan Medical Center (AMC) cohort patients, along with clinical annotations, have been deposited in the NCBI Gene Expression Omnibus and are accessible through GEO Series Accession Number GSE212810. Transcriptome data sets and pathologic responses to NAC for the three external cohorts were retrieved from the Gene Expression Omnibus database under accession numbers GSE48277 (MDA MVAC), GSE69795 (MDA DDMVAC), and GSE87304 (NAC metadata). Pathologic response records for the “NAC metadata” cohort were provided by Dr. Peter C. Black (University of British Columbia, Canada). Detailed information about the gene sets used for AMC validation and GSEA analysis of the external cohorts is shown in Table 1. A detailed list of GO categories and their corresponding genes in the functional analysis of transcriptome data sets with respect to gene networks, biological functions, and canonical pathways is shown in Table 2 below.

The original model for developing a machine learning classifier to distinguish between tumor epithelium and stroma for digital pathology analysis of the AMC cohort is shown in Table 2 below. Expression data for each protein in the tumor and stroma compartments, analyzed by digital pathology using the tumor/stroma classifier are also shown in Table 2 below along with relevant clinical annotations. In addition, the source code and constructed tumor/stroma classifier used in the study can be found at https://doi.org/10.5281/zenodo.7021255.

TABLE 1

| | | |
|---|---|---|
| NAME | SOURCE | IDENTIFIER |

Antibody

| | | |
|---|---|---|
| ANPEP (CD13) | Santa cruz | sc13536 |
| BDNF | Invitrogen | PA595183 |
| BICDL1 (CCDC64) | Invitrogen | PA566367 |
| CBS | Origenc | TA308071 |
| CD11c | NOVUS | NBP2-11599 |
| CD247 (CD3-zeta) | Santa cruz | sc1239 |
| CD44v6 | Abcam | ab78960 |
| CDK1 | Abcam | ab131450 |
| CK5/6 | DAKO | M7237 |
| CK20 | DAKO | M7019 |
| EMX1 | Santa cruz | sc398115 |
| EOGT | Invitrogen | PA553990 |
| FOXA1 | Santa cruz | sc101058 |
| FZD9 | Invitrogen | PA527119 |
| GAD2 | Santa cruz | sc377145 |
| GATA3 | Cell marque | 390M-16 |
| GCLC | Abcam | ab190685 |
| GCLM | Abcam | ab81445 |
| GGT7 | Invitrogen | PA553035 |
| GLS1 | Abcam | ab156876 |
| GSR | Abcam | ab128933 |
| ICAM1 | Invitrogen | MA5407 |
| IL15RA | NSJ Bioreagents | R32936 |
| IL27B (EBI3) | Invitrogen | MA524805 |
| KIFC2 | NOVUS | NBP2-17061 |
| KIR3DL1 | Invitrogen | PA5102443 |
| MITF | Invitrogen | PA5-82074 |
| Non-phosphorylated | Cell signaling | 4270S |
| β-Catenin | | |
| PLOD2 | Protein tech | 21214-1-AP |
| PPP2R5A | Santa cruz | sc271151 |
| PRDX1 | Abcam | ab15571 |
| PSAT1 | NOVUS | NBP1-32920 |
| QARS1(GlnRS) | Santa cruz | sc271078 |
| RPAP1 | Invitrogen | PA559703 |
| RPL9 | Santa cruz | sc100828 |
| SALLA | Biocare | CM384A,C |
| SOX2 | Abcam | ab92494 |
| TFCP2L1-middle | Aviva | OAAB09683 |
| TRAF2 | Santa cruz | sc7346 |
| TRAF6 | Santa cruz | sc8109 |
| β-Catenin | Santa cruz | sc7199 |

Oligonucleotide

| | | |
|---|---|---|
| Human ANPEP_F | CGGGCCACGGCGATT | SEQ ID NO: 1 |
| Human ANPEP_R | GGAGTGGGTAGGGTGTGTCATAA | SEQ ID NO: 2 |
| Human BDNF_F | CTACGAGACCAAGTGCAATCCC | SEQ ID NO: 3 |
| Human BDNF_R | AATCGCCAGCCAATTCTCTTT | SEQ ID NO: 4 |
| Human BICDL1_F | AGCTGCTCACAACCGATTCA | SEQ ID NO: 5 |
| Human BICDL1_R | GTACATTCTTGCCGCGCATC | SEQ ID NO: 6 |
| Human B2MG_F | GAGGGCTGGCAACTTAGAGG | SEQ ID NO: 7 |
| Human B2MG_R | ACAAGCTTTGAGTGCAAGAGA | SEQ ID NO: 8 |
| Human CBS_F | CCCCCTGGCTCACTACGA | SEQ ID NO: 9 |
| Human CBS_R | CACTGAAGCCACCAGCATGT | SEQ ID NO: 10 |
| Human CD11c_F | CCGACCATATCTGCCAGGAC | SEQ ID NO: 11 |
| Human CD11c_R | GCCCTTCAGGGTGAAATCCA | SEQ ID NO: 12 |
| Human CD247_F | GCCAGAACCAGCTCTATAACGA | SEQ ID NO: 13 |
| Human CD247_R | GGCCACGTCTCTTGTCCAA | SEQ ID NO: 14 |
| Human CD44_F | CTGCCGCTTTGCAGGTGTA | SEQ ID NO: 15 |
| Human CD44_R | CATTGTGGGCAAGGTGCTATT | SEQ ID NO: 16 |
| Human CDK1_F | AAACTACAGGTCAAGTGGTAGCC | SEQ ID NO: 17 |
| Human CDK1_R | TCCTGCATAAGCACATCCTGA | SEQ ID NO: 18 |
| Human CK14_F | TGAGCCGCATTCTGAACGAG | SEQ ID NO: 19 |
| Human CK14_R | GATGACTGCGATCCAGAGGA | SEQ ID NO: 20 |
| Human CK20_F | GGACGACACCCAGCGTTTAT | SEQ ID NO: 21 |
| Human CK20_R | CGCTCCCATAGTTCACCGTG | SEQ ID NO: 22 |
| Human CK5_F | CCAAGGTTGATGCACTGATGG | SEQ ID NO: 23 |
| Human CK5_R | TGTCAGAGACATGCGTCTGC | SEQ ID NO: 24 |
| Human CTNNB1_F | CATCTACACAGTTTGATGCTGCT | SEQ ID NO: 25 |
| Human CTNNB1_R | GCAGTTTTGTCAGTTCAGGGA | SEQ ID NO: 26 |
| Human EMX1_F | CACGAAGCAGGCCAATGG | SEQ ID NO: 27 |
| Human EMX1_R | CTCTGCCCTCGTGGGTTTGT | SEQ ID NO: 28 |
| Human FOXA1_F | GCAATACTCGCCTTACGGCT | SEQ ID NO: 29 |
| Human FOXA1_R | TACACACCTTGGTAGTACGCC | SEQ ID NO: 30 |
| Human FZD9_F | TGCGAGAACCCCGAGAAGT | SEQ ID NO: 31 |
| Human FZD9_R | GGGACCAGAACACCTCGAC | SEQ ID NO: 32 |
| Human GAD2_F | ATTGGGAATTGGCAGACCAAC | SEQ ID NO: 33 |
| Human GAD2_R | TTGAAGTATCTAGGATGCCCTGTG | SEQ ID NO: 34 |
| Human GATA3_F | GCCCCTCATTAAGCCCAAG | SEQ ID NO: 35 |
| Human GATA3_R | TTGTGGTGGTCTGACAGTTCG | SEQ ID NO: 36 |
| Human GCLC_F | GGAGGAAACCAAGCGCCAT | SEQ ID NO: 37 |
| Human GCLC_R | CTTGACGGCGTGGTAGATGT | SEQ ID NO: 38 |
| Human GCLM_F | TGTCTTGGAATGCACTGTATCTC | SEQ ID NO: 39 |
| Human GCLM_R | CCCAGTAAGGCTGTAAATGCTC | SEQ ID NO: 40 |
| Human GGT7_F | CTACAGTACAGCCAGGCAGG | SEQ ID NO: 41 |
| Human GGT7_R | GAACTGGAAGACAAGGCCCA | SEQ ID NO: 42 |
| Human GLS1_F | GCATTCCTGTGGCATGTATGACTT | SEQ ID NO: 43 |
| Human GLS1_R | CCCCCAGCAACTCCAGATTT | SEQ ID NO: 11 |
| Human GPX1_F | CCAGTTTGGGCATCAGGAGA | SEQ ID NO: 45 |
| Human GPX1_R | AGCATGAAGTTGGGCTCGAA | SEQ ID NO: 46 |
| Human GPX2_F | GACTTCACCCAGCTCAACGA | SEQ ID NO: 47 |
| Human GPX2_R | ATGCTCGTTCTGCCCATTCA | SEQ ID NO: 48 |
| Human GPX4_F | CAGTGAGGCAAGACCGAAGT | SEQ ID NO: 49 |
| Human GPX4_R | CGGTGTCCAAACTTGGTGAAG | SEQ ID NO: 50 |
| Human GSR_F | TTCCAGAATACCAACGTCAAAGG | SEQ ID NO: 51 |
| Human GSR_R | GTTTTCGGCCAGCAGCTATTG | SEQ ID NO: 52 |
| Human HS3ST6_F | ATCTCCGACTACGCCCAGAC | SEQ ID NO: 53 |
| Human HS3ST6_R | CGTGACGACCCGTTTCAGG | SEQ ID NO: 54 |
| Human ICAM1_F | CGGCTGACGTGTGCAGTAATA | SEQ ID NO: 55 |
| Human ICAM1_R | GGCGCCGGAAAGCTGTA | SEQ ID NO: 56 |
| Human IL15RA_F | CCCAGCTCAAACAACACAGC | SEQ ID NO: 57 |
| Human IL15RA_R | AGGTAGCATGCCAGGAGAGA | SEQ ID NO: 58 |
| Human IL27B_F | ATCCGTTACAAGCGTCAGGG | SEQ ID NO: 59 |
| Human IL27B_R | TCCCCGTAGTCTGTGAGGTC | SEQ ID NO: 60 |
| Human KIFC2_F | CGCCTTTTACTCGTTGCTCA | SEQ ID NO: 61 |
| Human KIFC2_R | CTGTCAACAGTGAGGGGACC | SEQ ID NO: 62 |
| Human KIR3DL1_F | CCAGGTCCCCTGGTGAAATC | SEQ ID NO: 63 |
| Human KIR3DL1_R | CGCTGTTGGCTGTTCTGTTC | SEQ ID NO: 64 |
| Human MITF_F | AGGGAGCTCACAGCGTGTAT | SEQ ID NO: 65 |
| Human MITF_R | AGGTCTTGGCTGCAGTTCTC | SEQ ID NO: 66 |
| Human PLOD2_F | CATGGACACAGGATAATGGCTG | SEQ ID NO: 67 |
| Human PLOD2_R | AGGGGTTGGTTGCTCAATAAAAA | SEQ ID NO: 68 |
| Human PPP2R5A_F | TGCCAATTATGTTTGCCAGTTT | SEQ ID NO: 69 |
| Human PPP2R5A_R | TCCATTAGGGTTTTCAGCACATT | SEQ ID NO: 70 |
| Human PRDX1_F | CATTCCTTTGGTATCAGACCCG | SEQ ID NO: 71 |
| Human PRDX1_R | CCCTGAACGAGATGCCTTCAT | SEQ ID NO: 72 |
| Human PSAT1_F | GGCCAGTTCAGTGCTGTCC | SEQ ID NO: 73 |
| Human PSAT1_R | GCTCCTGTCACCACATAGTCA | SEQ ID NO: 74 |
| Human QARS1_F | CGGCGTCTCTCCTTCCTTGT | SEQ ID NO: 75 |
| Human QARS1_R | TACTCAAGGGCAGCGCTTAGC | SEQ ID NO: 76 |
| Human RPAP1_F | TCCTGGGAGCAGGTTGTTTG | SEQ ID NO: 77 |
| Human RPAP1_R | AGACCTTCAGAAGCCCCAGA | SEQ ID NO: 78 |
| Human RPL9_F | TGCTCACTTCCCCATCAACG | SEQ ID NO: 79 |
| Human RPL9_R | GCTTGCTGAATCAAAGCCG | SEQ ID NO: 80 |
| Human SALL4_F | TGCGGAGTCTGTGGTGTACCTA | SEQ ID NO: 81 |
| Human SALLA_R | GATTCACCGCCACCTTGG | SEQ ID NO: 82 |
| Human SIRT6_F | GCAGTCTTCCAGTGTGGTGT | SEQ ID NO: 83 |
| Human SIRT6_R | GATAGAGCCGTTGATCCGGG | SEQ ID NO: 84 |
| Human SOX2_F | AACCAGCGCATGGACAGTTA | SEQ ID NO: 85 |
| Human SOX2_R | GACTTGACCACCGAACCCAT | SEQ ID NO: 86 |
| Human TFCP2L1_F | GCTCTTCAACGCCATCAAA | SEQ ID NO: 87 |
| Human TFCP2L1_R | CAGGGGCACTCGATTCTG | SEQ ID NO: 88 |
| Human TRAF2_F | GGAGGCATCCACCTACGATG | SEQ ID NO: 89 |
| Human TRAF2_R | GGGAGAAGATGGCGGGTATG | SEQ ID NO: 90 |
| Human TRAF6_F | CTGCTTGATGGCATTACGAGAA | SEQ ID NO: 91 |
| Human TRAF6_R | TGCAGGCTTTGCAGAACCTA | SEQ ID NO: 92 |

TABLE 2
Biological sample

| Bladder cancer patient sample | This study | IRB; AMC |
|---|---|---|
| | | (2020- |
| | | 0064) |

Critical commercial analysis

| | | |
|---|---|---|
| SMARTer Stranded Total RNA- | Takara | 634411 |
| Seq Kit v2 | | |
| Taqman Reverse Transcription | Applied | N8080234 |
| Reagents | Biosystems | |
| ultraView Universal DAB | Roche | 760-500 |
| detection kit | | |

Deposited data

| | | |
|---|---|---|
| Transcriptome data set for AMC | This paper | GSE212810 |
| cohort | | |
| Transcriptome data set for MDA | Choi et al., | GSE48277 |
| MVAC cohort | 2014 | |
| MDA DDMVAC cohort | McConkey et | GSE69795 |
| | al., 2016 | |
| Transcriptome data set for NAC | Seiler et al., | GSE87304 |
| metadata cohort | 2017 | |

Software and Algorithms

| | | |
|---|---|---|
| GraphPad Prism 7.0 | GraphPad | N/A |
| | Software | |
| MetaCore | Clarivate | N/A |
| | Analytics | |
| GSEA | Broad Institute | N/A |
| ExDEGA | Ebiogen Inc. | N/A |
| QuPath | Bankhead et | N/A |
| | al., 2017 | |
| Manuscript analysis workflow | This study | https://doi.org/ |
| | | 10.5281/ |
| | | zenodo.7021255 |

Other

| | | |
|---|---|---|
| BenchMark XT Automated IHC/ISH | Roche | N/A |
| slide staining system | | |
| Vectra 3.0 Automated Quantitative | Perkin-Elmer | CLS142568 |
| Pathology Imaging System | | |
| Clinical annotations | This study | Table S10 |
| | | and S11 |
| Cryostat | Leica | RM2125 RTS |

### 1-2. Patient Sample Collection and Cohort Organization

This study was approved by the AMC Institutional Review Board (2020-0064). The present invention included 63 consecutive MIBC (cT2-4aNOM0) patients who underwent TUR at AMC between August 2011 and August 2017, followed by NAC, and subsequently curative surgery using available FFPE tumor tissue. All the patients were designated as a validation cohort because their pathologic response could be assessed using surgical specimens. Among the 63 patients, 20 patients who were recorded as “incomplete TUR” and showed a striking contrast in pathologic response were selected as a discovery cohort. Patients' clinical information, including NAC, surgical TUR history, tumor progression, and survival time, were obtained from electronic medical records.

All pathological data of the present invention were reviewed by an urologic pathologist (Y.M.C.) and reevaluated according to the ‘2016 World Health Organization Classification of Tumors’ and staged according to the ‘American Joint Committee on Cancer Staging System, 8th edition’. NAC response was defined as downstaging to ypT1 or lower in the absence of invasive tumor or lymph node metastases (ypN0) on pathologic examination of radical or partial cystectomy specimens. Lack of pathologic response was defined as residual tumor or lymph node metastases invading beyond the muscularis propria (≥ypT2 and/or ypN0).

### 1-3. Laser Capture Microdissection (LCMD) and RNA Isolation

LCMD was performed on hematoxylin-eosin stained tissue slides using a Leica LMD6500 laser capture dissecting microscope (Leica Microsystems, Deerfield, IL, USA) as described by the manufacturer. The urothelial carcinoma area was microdissected carefully, as close as possible to the tumor cells, to avoid contamination of stromal and inflammatory cells and to exclude necrotic tumor areas. Total RNA from LCMD samples was isolated using the RNeasy FFPE kit (73504, QIAGEN, Valencia, CA, USA). RNA quality was assessed with an Agilent 2100 bioanalyzer using an RNA 6000 Nano Chip (G2939BA, Agilent Technologies, Amstelveen, The Holland), and RNA quantification was performed using an ND-2000 Spectrophotometer (Thermo Fisher Scientific, Waltham, MA, USA).

### 1-4. Library Preparation and Sequencing

Library construction from isolated RNA was performed using the QuantSeq 3′ mRNA-Seq Library Prep Kit (Lexogen, Inc., Austria) according to the manufacturer's instructions. Briefly, total RNA for each sample was prepared, and oligo-dT primers containing an Illumina-compatible sequence at the 5′ end were hybridized to the RNA and reverse transcription was performed. After the RNA template was degraded, second-strand synthesis was initiated by a random primer containing an Illumina-compatible linker sequence at the 5′ end. The double-stranded library was purified using magnetic beads to remove all reaction components. The library was amplified to add the full adapter sequence required for cluster creation. The completed library was purified from the PCR components. High-throughput sequencing was performed by single-end 75 sequencing using NextSeq 500 (Illumina, Inc., San Diego, CA, USA).

### 1-5. Analysis of Transcriptome Data Sets

QuantSeq 3′ mRNA-Seq reads were aligned using Bowtie2. The Bowtie2 index was generated from genome assembly sequences or representative transcriptome sequences for genome and transcriptome alignment. The alignment files were used for transcriptome assembly, abundance estimation, and differential gene expression detection. Differentially expressed genes were determined based on the number of unique and multiple alignments using the coverage of Bedtools. RC (Read Count) data were processed based on the quantile normalization method using the R EdgeR package (R development Core Team, 2020) using Bioconductor. Gene classification was based on searches performed in DAVID (//david.abcc.ncifcrf.gov/) and Medline databases (//www.ncbi.nlm.nih.gov/). Data mining and graphical visualization were performed using ExDEGA (Ebiogen Inc., Seoul, Korea).

### 1-6. MetaCore and GSEA Transcriptome Analysis

Functional transcriptome analysis was performed using MetaCore (Clarivate Analytics, Philadelphia, PA, USA) or GSEA (Broad Institute, Cambridge, MA, USA) software with default settings, which provide core analysis and enrichment of gene sets for gene networks, biofunctions, and canonical pathways, respectively. In MetaCore analysis, 1.5-fold up-or down-regulation with p<0.05 was defined as the cutoff value for significant change. Details of statistical values and significant genes for biological processes and pathways in MetaCore analysis are shown in Table 3. The gene sets used in GSEA were obtained from the functional gene set (C2) database, which was curated using classifiers based on published literature or multiple library sources. A significant difference was determined based on FDR (false discovery rate)<0.25.

TABLE 3

| | −log10 | |
|---|---|---|
| Maps | (p-value) | Network objects |
| Role of Nek in cell | 5.618 | Tubulin alpha, Nek8, Histone H3, |
| cycle_cell cycle | | NEK9, Histone H1, MAD2a, Ran |
| regulation | | |
| HCV-dependent cytoplasmic | 3.783 | ERK1/2, ERK2 (MAPK1), PKC, PKR, |
| signaling leading to HCC | | eIF2S1 |
| Regulation of STK3/4 | 3.673 | ERK1/2, Nek8, RASSF6, G-protein |
| (Hippo) pathway and | | alpha-q/11, HIPK2, ERK1 (MAPK3), |
| positive regulation of | | MASK |
| YAP/TAZ function | | |
| Gastrin in immune | 3.422 | ERK2 (MAPK1), G-protein alpha-q/11, |
| response_inflammatory | | G-protein alpha-q, ERK1 (MAPK3), |
| response | | TRAF6, PKC-delta, MAP2K5 (MEK5) |
| Signaling by immune | 3.316 | ERK1/2, ERK2 (MAPK1), MAPKAPK2, |
| response_BAFF | | TRIM2, ERK1 (MAPK3), PKC-delta |
| ATM/ATR regulation of DNA | 3.316 | ERK2 (MAPK1), MAPKAPK2, Histone H3, |
| damage_G2/M checkpoint: | | 14-3-3, PP2A regulatory, ATM |
| cytoplasmic signaling | | |
| Lysophosphatidic acid | 3.270 | ERK1/2, TRIP6, TRAF6, PKC, PKC- |
| signaling via immune | | delta, ICAM1 |
| response_NF-κB | | |
| IFN-alpha/beta signaling | 3.233 | ERK1/2, Filamin B (TABP), MAPKAPK2, |
| via immune response_MAPK | | ISG15, PRMT1, PKC-delta, PKR |
| Role of PKR in apoptosis | 3.225 | ERK1/2, PPP2R5A, PP2A regulatory, |
| and survival_stress- | | TRAF6, PKR, cIF2S1 |
| induced apoptosis | | |
| Gastrin for gastric | 3.066 | ERK1/2, TFF1, G-protein alpha-q/11, |
| mucosal differentiation | | G-protein alpha-q, PKC |

### 1-7. Tissue Microarray (TMA) Construction and Immunohistochemical (IHC) Analysis

TMA blocks were constructed from FFPE tissue blocks of TUR specimens harvested prior to NAC using tissue microarrays (Beecher Instruments, Silver Spring, MD, USA). To overcome intratumoral heterogeneity, three representative cores of 0.6 mm diameter were included from different regions of each tumor. When a tumor contained areas of different histologic grades and stages, a TMA was generated from the tumor area of the highest grade and stage.

THC staining was performed on TMA constructs using an automated staining system (BenchMark XT, Ventana Medical Systems, Tucson, AZ, USA) and the ultraView Universal DAB detection kit (Ventana Medical Systems). The nuclei were counterstained with hematoxylin. Detailed antibody information and staining conditions are shown in Tables 1 and 2 above.

### 1-8. Computational Analysis of IHC Staining Images

IHC slides were scanned at ×20 magnification at a resolution of 0.22 mm per pixel using a Panoramic 250 Flash slide scanner (3D HISTECH, Budapest, Hungary) and they were subjected to computer analysis using QuPath, an open-source software platform for biomedical image analysis.

To individually assess the intensity of expression in the epithelial and stromal compartments, the present inventors developed a machine learning classifier to distinguish tumor epithelial cells from stroma in immunohistochemical images stained with 3,3′-diaminobenzidine (DAB) and counterstained with hematoxylin. First, representative IHC slide images were selected to construct training, test, and validation sets (CBS and CD73 from the discovery cohort, and CD11c and CDK1 from the advanced BC cohort not analyzed in this study). The images were opened and annotated with tumor epithelial cells and stroma regions using QuPath. The annotated regions were segmented according to the cell detection function of QuPath using default parameters. The QuPath feature is intended to identify cells in a specific region. To classify the detected cells using a deep learning-based approach, the present inventors wrote a QuPath script that extracts a patch of size 224×224 pixels centered on the cell center of each of the IHC slides. As a result, 224,101 patches were obtained, labeled as either “tumor” (96,149 patches) or “stroma” (127,772 patches). Using the StratifiedShuffleSplit method from Scikit-learn, a Python library package for machine learning, the patches were randomly split into a training set (40%), a validation set (30%), and a test set (30%) while preserving the proportion of “tumor” and “stroma.” The present inventors built a model by training a pre-trained model provided by PyTorch, an open source machine learning framework. A model with an accuracy of 0.96 was obtained using ResNet34, a state-of-the-art convolutional neural network model for image classification starting with pre-trained parameters.

For inference, cell patches named with a unique ID number were extracted from each IHC slide. A deep learning model was applied to classify the patches and a Python script was written to export a table consisting of cell IDs and classification results as “tumor” or “stroma.” A QuPath script was written to load the table and visualize the inference results. Model building, including annotations of tumor epithelial cells and stroma, was performed by a pathologist (S.Y.Y.), but the classification results were validated by another pathologist (J.A.J.). Where necessary, incorrectly classified regions were appropriately corrected.

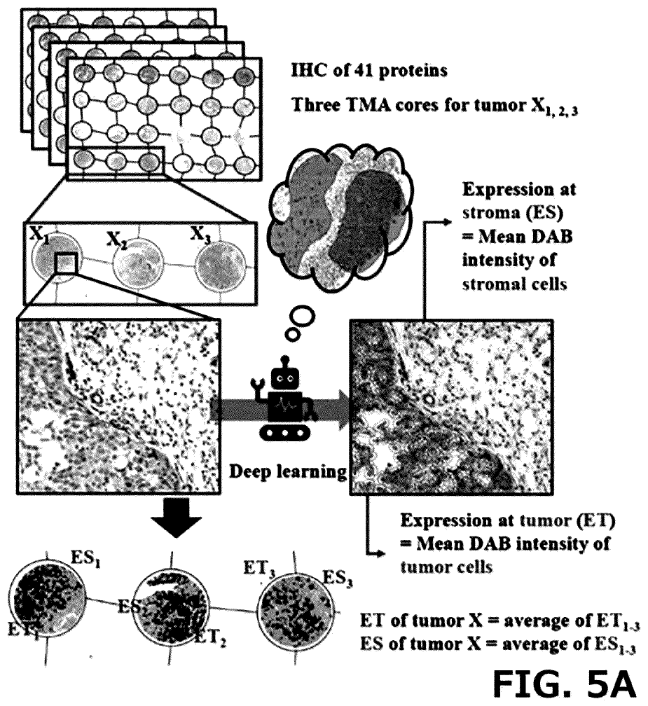

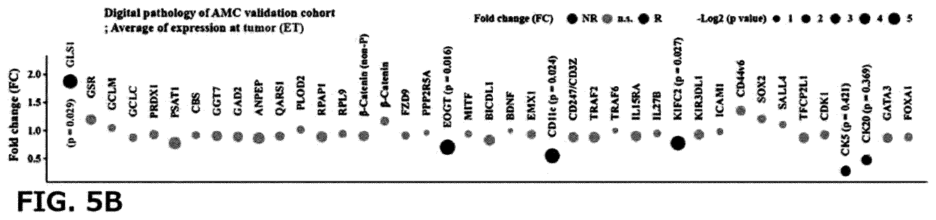

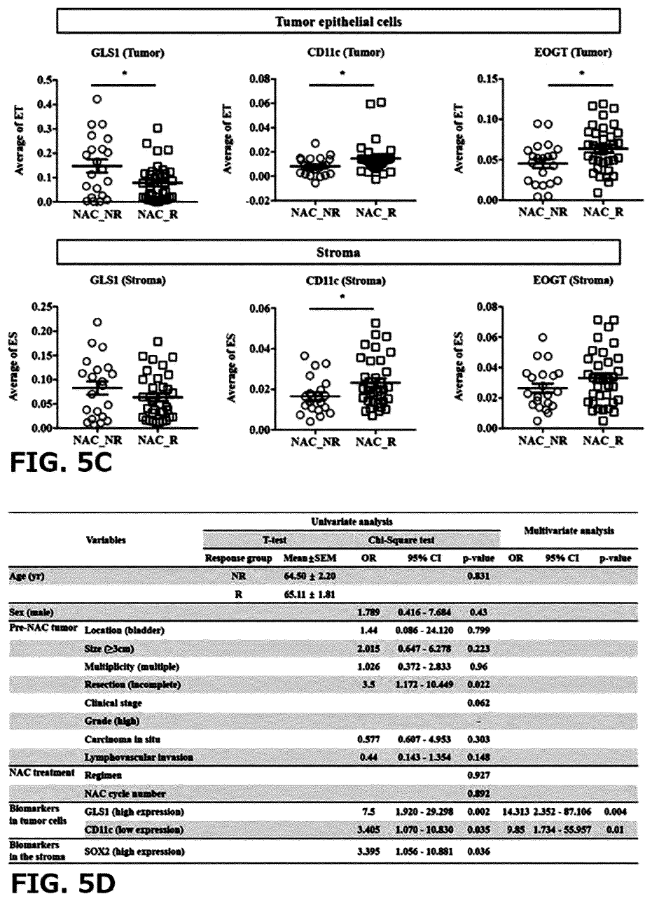

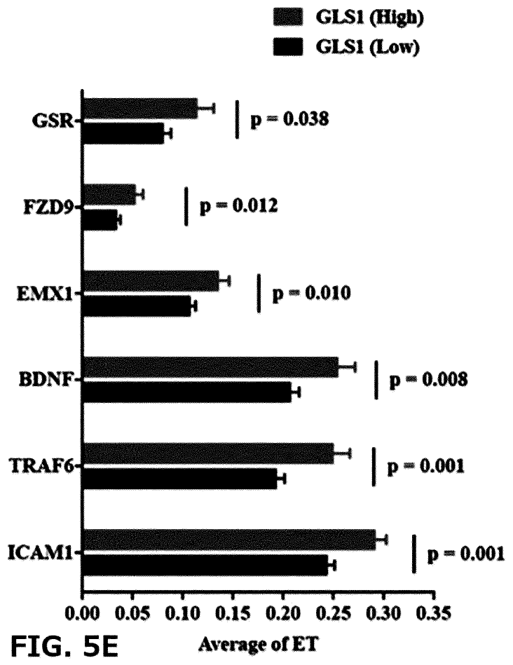

To quantify protein expression levels, DAB staining intensity for each cell was exported from QuPath as the measurement ‘Cell: DAB OD mean’. For TMA cores, protein expression levels in tumor epithelium and stroma were calculated separately by averaging the DAB intensities of cells classified as “tumor” and “stroma,” respectively. Since there were three TMA cores per tumor, the average results of the three cores were subsequently obtained to represent the tumor expression level (FIG. 5A).

### 1-9. Clinical MIBC Patient Cohort Analysis

Clinicopathological analysis of MIBC patients in the AMC cohort was performed using SPSS version 21.0. All continuous data were compared using Student's t-test, and categorical data were compared using Pearson's Chi-square, Fisher's Exact, and Kruscal-Wallis tests. Categorical IHC expression data were classified into high versus low expression of the indicated biomarkers based on cutoff values determined by a heuristic method. Univariate analysis of NAC response was performed to determine the clinical significance of each protein expression and clinicopathologic parameter. Statistically significant variables in univariate analysis were analyzed using multivariate analysis using binary logistic regression analysis. Independent variables were selected through backward stepwise selection, and a p-value<0.05 was considered statistically significant.

### 1-10. Quantification and Statistical Analysis

All quantitative data in the present invention are expressed as mean±standard error of the mean (SEM). Statistical significance for three or more groups was determined using one-way analysis of variance (ANOVA) with Bonferroni post-test. Statistical significance between the two groups was analyzed using unpaired Student's t-test. Statistical analyses were performed using GraphPad Prism 7.0 (GraphPad Software, La Jolla, CA, USA) or SPSS version 21.0 software. Significance was assumed for *p<0.05, **p<0.01, ***p<0.001.

### 1-11. Additional Resources

All quantifications of raw data and statistical analyses, including tests used, sample sizes and exact p-values, are shown in Tables 1 and 2 above. Raw digital pathology data for tumor and stromal compartments are provided along with nomenclature of patient cases used for RNA-seq analysis and associated clinical annotation. The source code for the digital pathology and tumor/stroma classifier construction is available at https://doi.org/10.5281/zenodo.7021255. DOI (digital object identifier) is listed in the main resource table.

### Example 2

### Results

### 2-1. Transcriptome Profiling Based on MIBC NAC Response

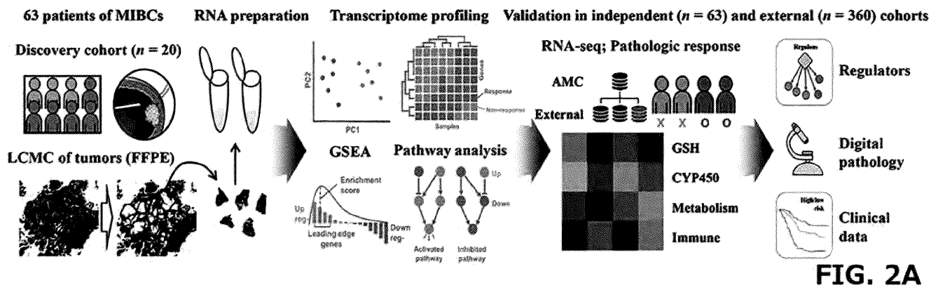

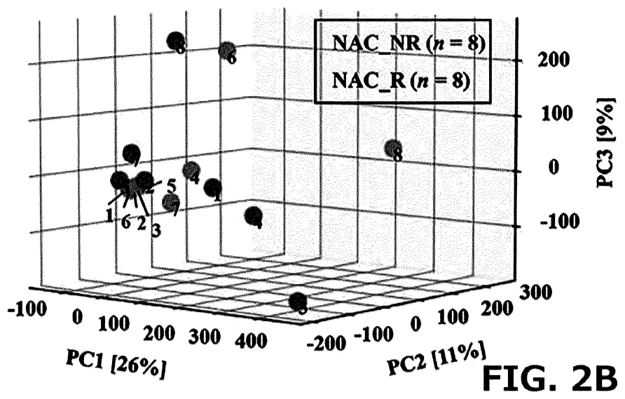

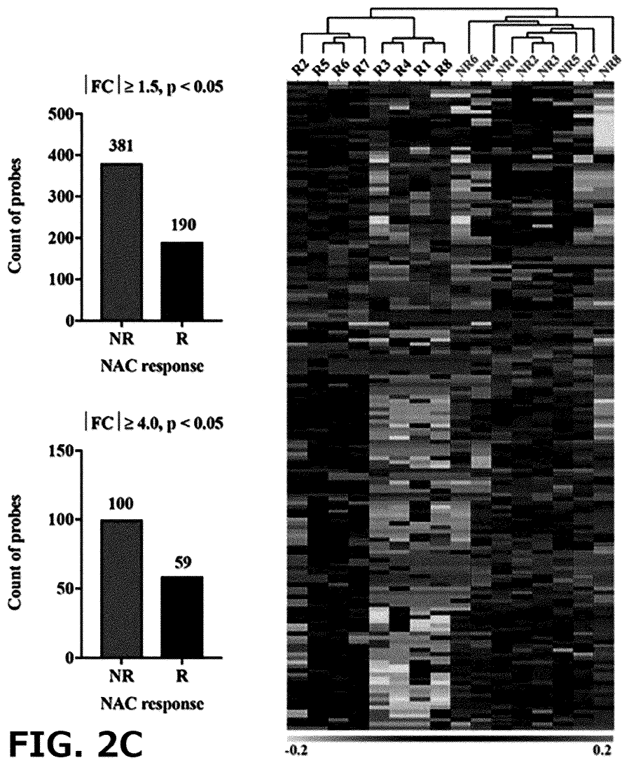

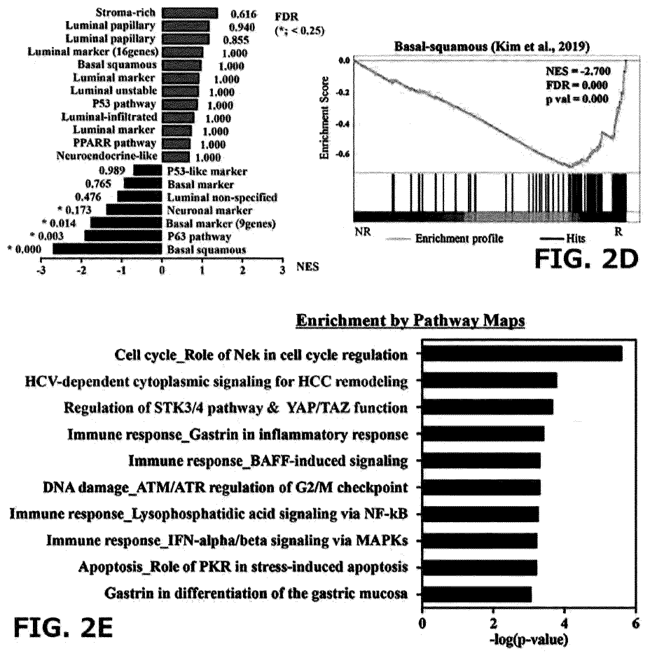

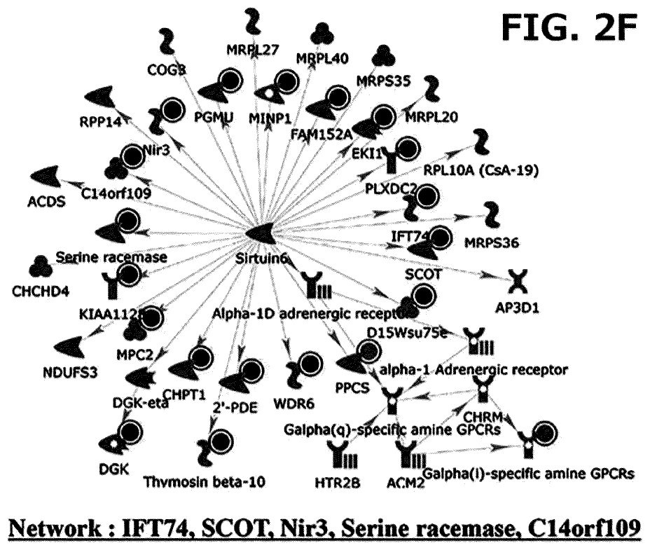

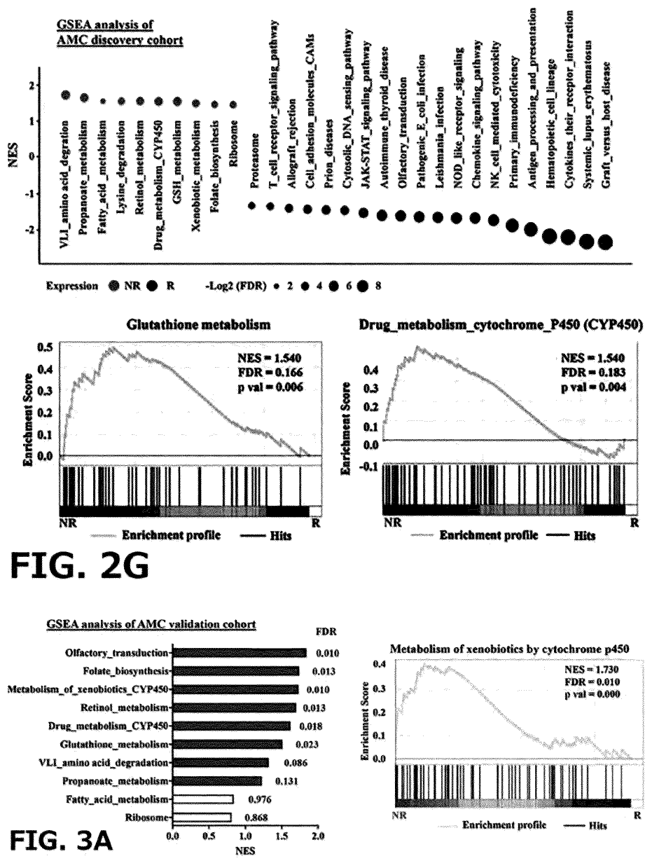

We retrospectively collected samples from 63 patients with MIBC (cT2-4aNOM0) who underwent transurethral resection (TUR) and NAC followed by radical/partial cystectomy (Table S1). Of these cases, 20 patients had “incomplete TURBT” recorded in their TURBT surgical records and were assigned to the AMC discovery cohort, indicating residual tumor remaining after TUR, which ruled out the possibility of complete tumor resection by TUR prior to NAC and allowed for evaluation of treatment response to NAC. The discovery cohort was divided into two groups with significant differences in NAC response: 8 NAC responders (R) with no residual invasive tumor in radical/partial cystectomy specimens, and 12 NAC non-responders (NR) with large residual MIBC (4.9±2.5 cm). To investigate the tumor-specific molecular signature of the NAC response without stroma, we extracted 16 patients (n=8/group, NR and R) from the discovery cohort and compared their genome-wide transcriptome profiles (FIG. 2A). Principal component analysis (PCA) showed that the overall transcriptome showed little difference between the NR and R groups (FIG. 2B). Using the cutoff values of 1 fold change|>1.5 and p<0.05, we identified 571 differentially expressed genes (DEGs), of which 381 were up-regulated and 190 were down-regulated in the NR group compared with the R group (FIG. 2C).

The present inventors performed gene set enrichment analysis (GSEA) to investigate whether the transcriptomes of the NR and R groups correlate with published molecular classifications defining MIBC molecular subtypes. Consistent with previous findings, NAC-sensitive tumors were enriched for basal and basal squamous (Ba/Sq) classifiers (FIG. 2D), as was the p63 pathway, which was elevated in the basal MIBC subtype. However, NAC-resistant tumors did not match the published MIBC molecular classifier.

Therefore, the tumor transcriptome of the AMC discovery cohort was further characterized for gene networks, biofunctions, and canonical pathways using MetaCore and GSEA analysis methods. Gene ontology (GO) analysis showed that tumors from NAC NR and R groups exhibited differential expression of genes involved in Hippo and YAP/TAZ function, cell cycle, inflammation and immune response pathways (FIG. 2E), and several metabolic processes. MetaCore gene network analysis showed that genes related to metabolic processes were enriched in NR group tumors, and immune processes were enriched in R group tumors (FIG. 2F).

Consistently, GSEA revealed that gene sets related to cell metabolism, including Valine_leucine_isoleucine (VLI) amino acid (AA) degradation (NES=1.72; FDR=0.143), cytochrome P450 (CYP) drug metabolism (NES=1.53; FDR=0.183), and GSH metabolism (NES=1.54; FDR=0.166), were significantly enriched in the transcriptome of NR group tumors (FIG. 2G). In contrast, most of the gene sets enriched in R group tumors were those involved in immune response (FIG. 2G), including cytokine (NES=−2.19; FDR=0.000) and chemokine (NES=−1.660; FDR=0.051) signaling and natural killer cell-mediated cytotoxicity (NES=−1723; FDR=0.036).

### 2-2. Confirmation of Upregulation of GSH-Related Metabolic Processes in NAC-Resistant MIBC

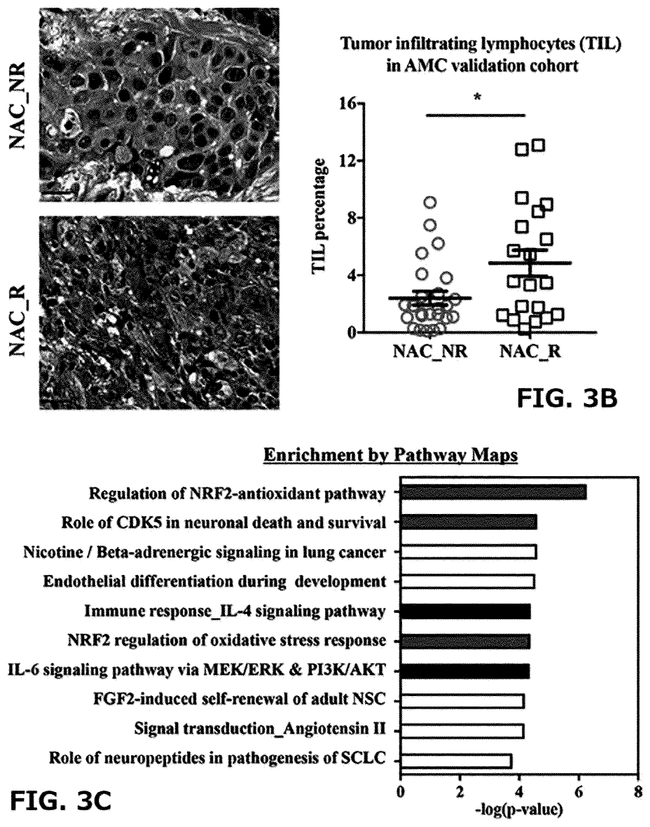

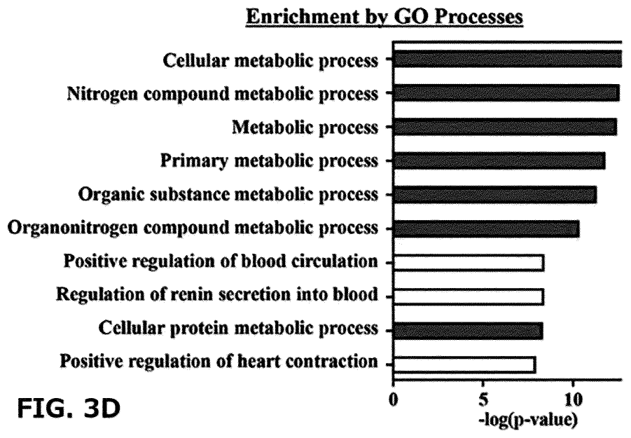

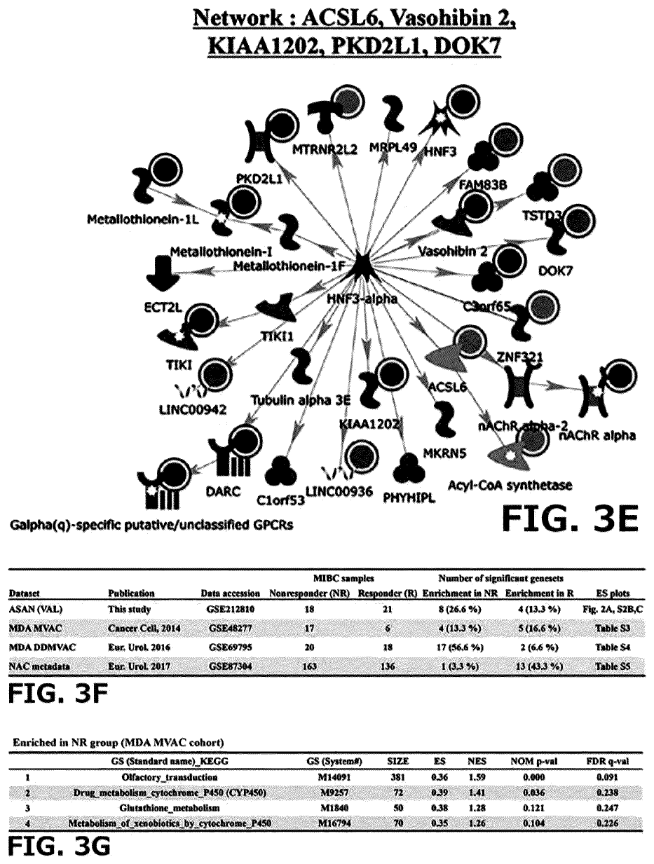

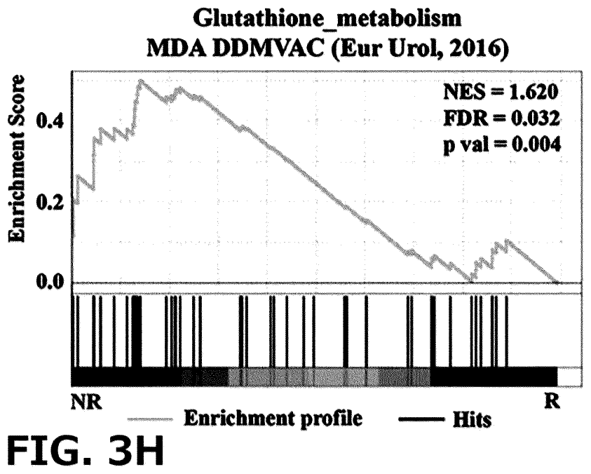

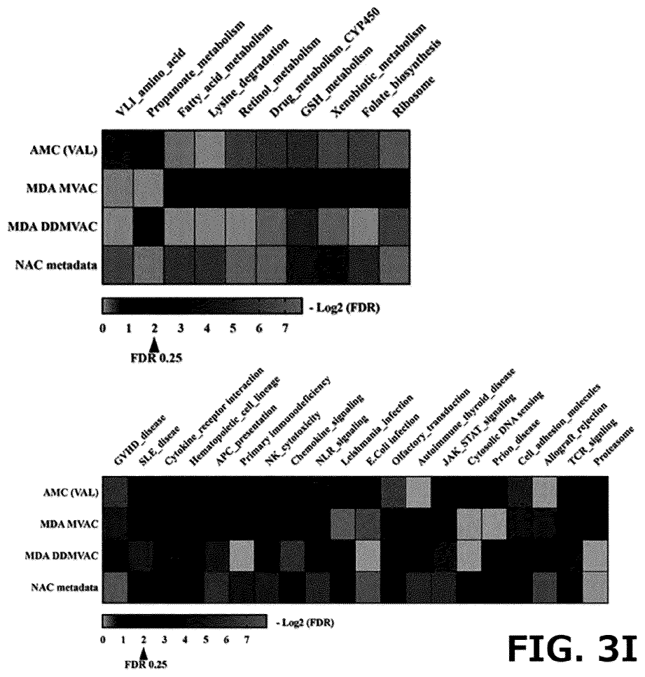

To further confirm the above results, the present inventors used 63 MIBC patients (cT2-1aNOM0) as a validation cohort. All the patients in the experiment underwent radical or partial surgery, allowing for evaluation of pathologic response. Pathologic response to NAC (≤ypT1 and ypN0) was observed in 37 patients (57.7%). Among 63 cases in the validation cohort, LCMD of tumors was performed on FFPE TURBT specimens, collecting transcriptome data sets of 39 patients (NR, n=18; R, n=21). When GSEA was performed using the 30-gene set to characterize the discovery cohort, NR tumors in the validation cohort were strongly enriched for gene sets associated with metabolic processes related to CYP or GSH metabolism and folate biosynthesis. It plays a role in regulating redox status by generating GSH (FIG. 3A). When GSEA was performed using the 30-gene set to characterize the discovery cohort, NR tumors in the validation cohort strongly expressed gene sets related to folate biosynthesis, which produces GSH and plays an important role in redox status regulation, as well as metabolic processes related to CYP or GSH metabolism (FIG. 3A). Tumors in group R of the validation cohort were characterized for cytokine and antigen presentation gene sets. Tumor infiltrating lymphocytes (TILs) were counted in TUBRT tumor specimens from the AMC validation cohort, showing that TILs were higher in R group tumors compared to NR group tumors (FIG. 3B).

Importantly, MetaCore analysis indicated that the antioxidant pathway mediated by nuclear factor erythroid-2 related factor-2 (NRF2) was significantly upregulated in NR group tumors (FIG. 3C). NRF2 is a key regulator of genes involved in antioxidant responses, including the GSH metabolic pathway. In addition, NAC-resistant tumors in the validation cohort were strongly expressed in a subset of cellular metabolic processes and gene networks associated with detoxification of inorganic compounds (FIGS. 3D and 3E). This indicates an important role for NRF2-mediated antioxidant, metabolic and detoxification pathways in determining NAC responses in MIBC.

Subsequently, the set of 30 genes differentially associated with NAC response in the discovery cohort was applied to three independent, publicly available gene expression profiling data sets of pre-NAC TURBT specimens containing records of pathologic response to NAC (FIG. 3F). External institute cohorts include: i) the “MDA MVAC cohort” consisting of 23 patients treated uniformly with MVAC in the context of a phase 3 clinical trial, ii) the “MDA DDMVAC cohort” consisting of 38 patients in a phase 2 trial of DDMVAC and bevacizumab, and iii) the “NAC metadata cohort” consisting of 299 patients from 7 institutions treated with various NAC regimens. Consistent with the results from the AMC validation cohort, CYP drug/xenobiotic metabolism and GSH metabolism gene sets were significantly enriched in NR group tumors in both the “MDA MVAC” and “MDA DDMVAC” cohorts (FIGS. 3G to 3I), further supporting the importance of GSH metabolism in preoperative chemotherapy sensitivity in MIBC. Similarly, several immune response processes were significantly enhanced in R group tumors of the “NAC metadata cohort” (FIG. 3I).

Overall, in accordance with our results, transcriptome profiling of MIBC patient tumors from four independent cohorts demonstrated that GSH-related metabolic responses may characterize the biology of NAC-responsive MIBC subtypes, and immune response pathways may characterize the biology of NAC-sensitive MIBC subtypes.

### 2-3. MIBC NAC Response Predictive Biomarker

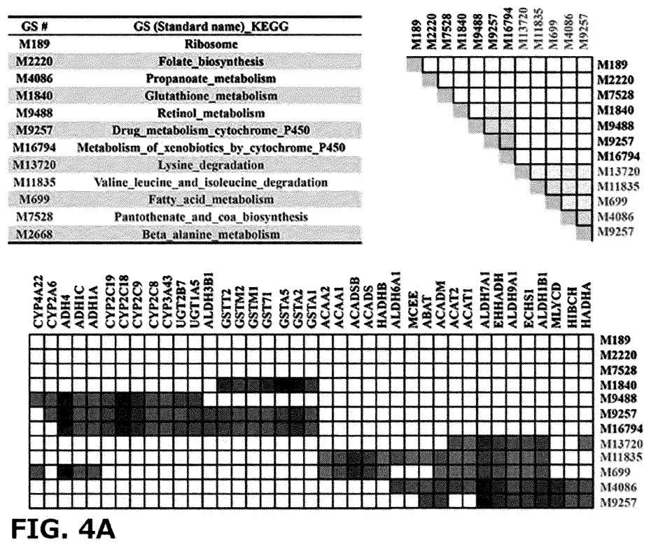

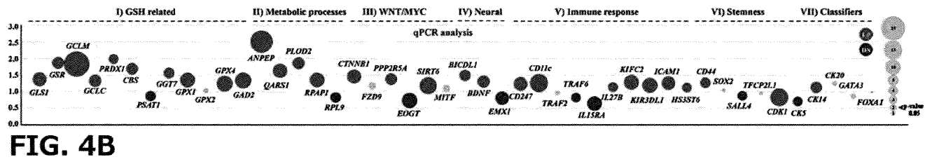

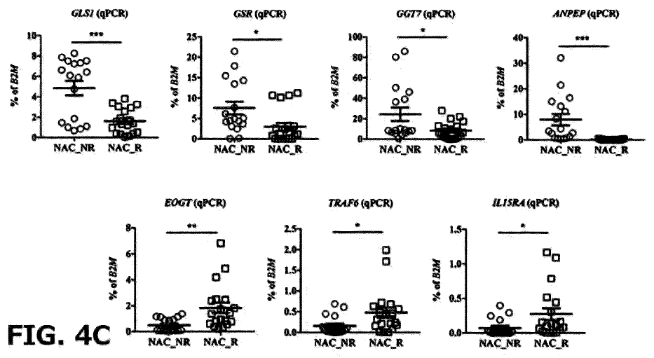

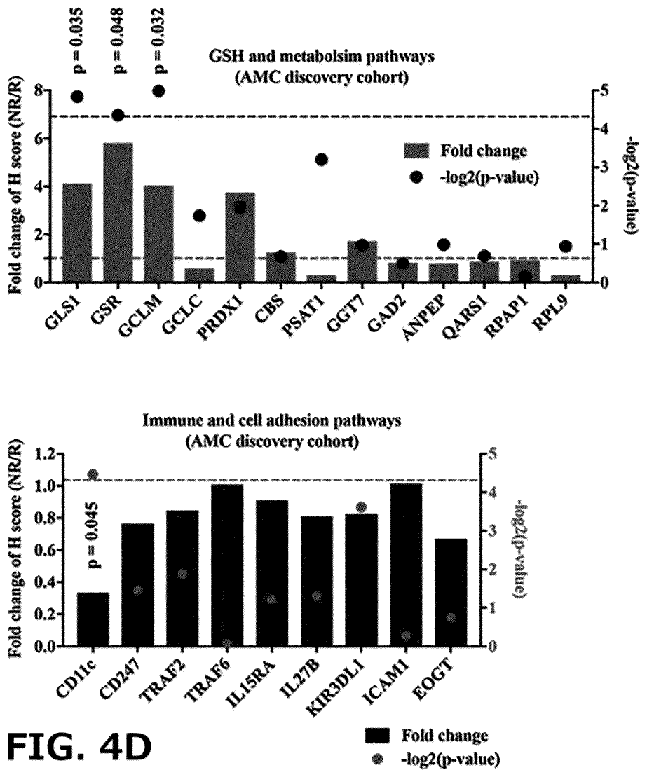

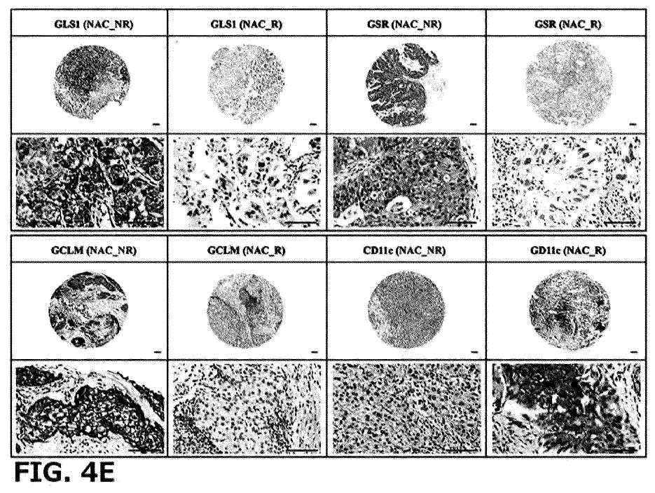

To identify potential biomarkers defining distinct molecular features of NAC response, the present inventors further explored gene network (MetaCore) and state-of-the-art (GSEA) analyses using transcriptome data sets from the AMC discovery cohort characterized by incomplete TURBT. MetaCore analysis revealed that several zinc finger protein (ZNF) family genes were upregulated in NR tumors, through networking with genes involved in translation elongation and termination, peptide biosynthesis process, and metabolism process. Similarly, GSEA state-of-the-art analysis revealed that several GSH S-transferase (GST) and CYP genes were highly expressed in NR tumors, as evidenced by clustering of GSH metabolism gene sets (FIG. 4A). NAC-sensitive MIBC exhibited coordinated activation of immune cell pathways and upregulation of cytokines and chemokines.

The present inventors then validated the expression of these putative biomarkers by quantitative PCR (qPCR) analysis of a cDNA library established using LCMD tumor specimens. Compared to R tumors, NR tumors showed marked expression of genes indicating: i) GSH metabolism, ii) metabolic processes, iii) WNT/MYC pathway, iv) neural markers, v) immune response, vi) cell adhesion, and vii) stem characteristics (FIG. 4B). Among these pathways, GSH building AA metabolism (GLS1 and CBS), synthesis (GCLM and GCLC), and utilization (GSR, GGT7, PRDX1, GPX1, and GPX4) were significantly enriched, suggesting their potential use as NAC-resistant MIBC biomarkers (FIG. 4C). In contrast, genes (TRAF6 and IL15RA) associated with immune response were activated in group R tumors.

### 2-4. Upregulation of GSH-Related Proteins in NAC-Resistant MIBCs

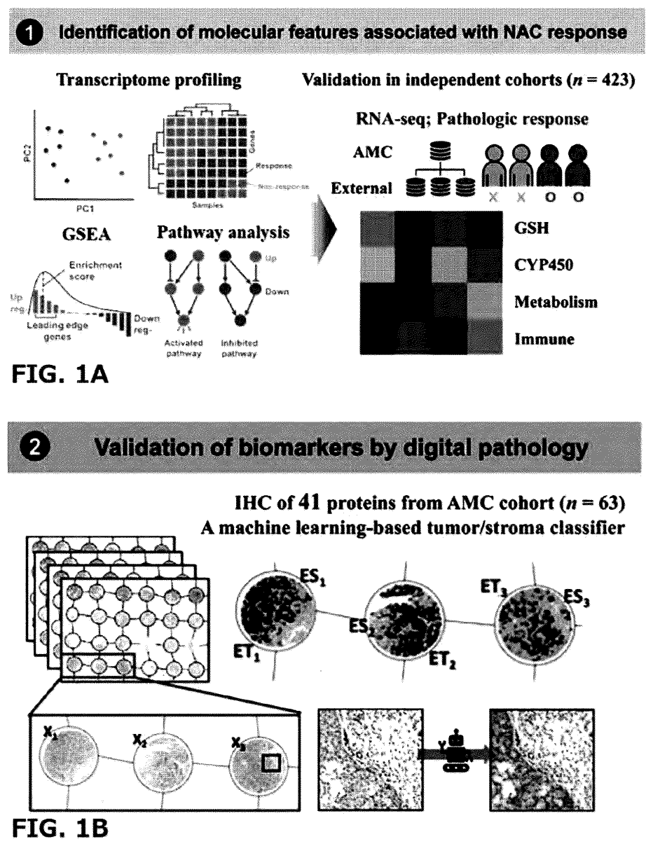

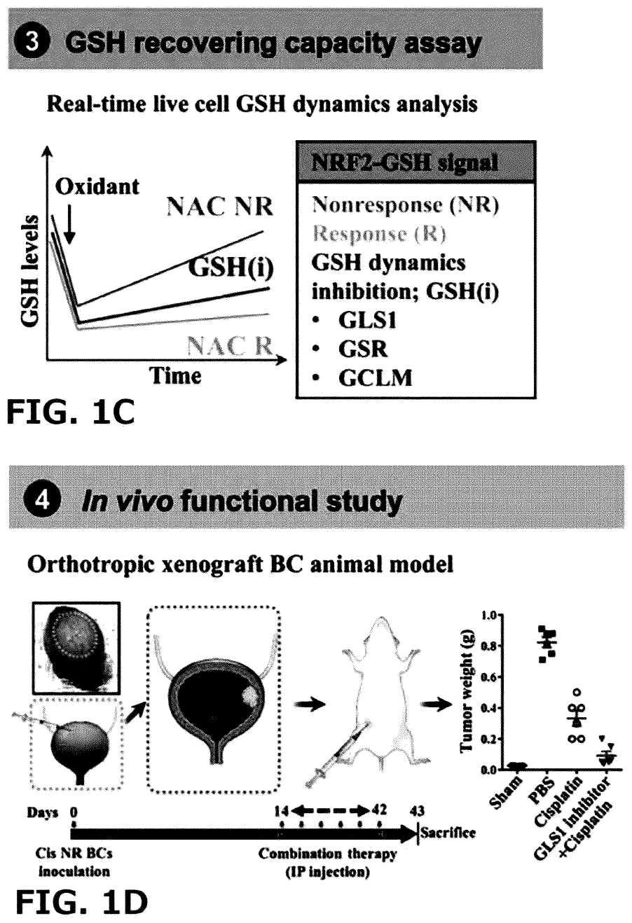

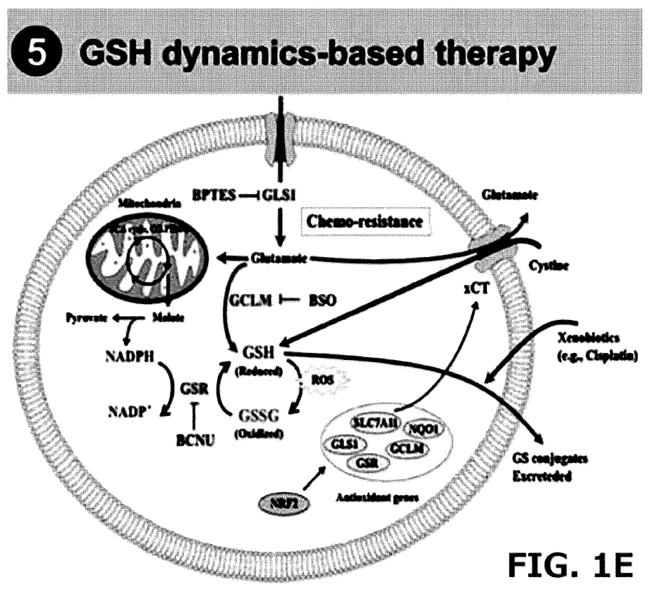

To investigate the potential clinical significance of these biomarkers validated at the transcriptome level, the present inventors examined the protein levels of the biomarkers using immunohistochemistry (IHC) on tissue microarray (TMA) constructs generated from pre-NAC TURBT specimens. Initially, the AMC discovery cohort was examined semiquantitatively by visual assessment by an uropathologist. Consistent with the transcriptome expression results above, GSH-related and immune-responsive proteins showed differential expression between NR and R TURBT samples (FIG. 1D). Among the affected proteins, GLS1, GSR, and GCLM proteins were significantly increased in NR tumors, whereas CD11c protein was decreased (FIG. 4E).

In addition, the present inventors digitized IHC slides from the AMC validation cohort to objectively measure protein levels and analyzed them with computerized pathology software. The present inventors sought to determine whether protein expression levels differ between tumor cell and stromal compartments. To this end, we built a deep learning model to segment IHC slide images into tumor epithelial cell or stromal compartments, and quantified the average intensity of the chromogen (3,3′-diaminobenzidine, DAB) separately for each compartment (FIG. 5A).

Importantly, high expression of GLS1 protein was also observed in NAC-resistant tumors from the AMC validation cohort, supporting the clinical significance of GLS1 in defining MIBC NAC response (FIGS. 5B and 5C). Consistent with the transcriptome profiling results, NAC-sensitive tumors showed elevated expression of proteins involved in immune response (CD11c) and cell adhesion (EOGT and KIFC2) (FIGS. 5B and 5C). Expression of CD11c and CD247/CD3Z proteins was decreased in the stroma of the NAC-sensitive group. In particular, the stroma of the NAC-resistant group showed significant upregulation of SOX2 protein, which contributes to the maintenance and progression of BCs.

### 2-5. Association Between Clinicopathologic Data and Biomarker Expression Data for Predicting NAC Response

Among the clinicopathological features and expression levels of various biomarkers, univariate analysis showed that NAC resistance was associated with incomplete resection of the tumor during TURBT (p=0.002), high levels of GLS1 protein (p=0.002), and low expression of CD11c on tumor cells (p=0.035) (FIG. 5D). High expression of SOX2 in the stroma compartment was also associated with NAC non-response in univariate analysis (p=0.036). In multivariate analysis, high expression of GLS1 (p=0.004) and low expression of CD11c (p=0.010) in tumor cells remained independent prognostic factors for predicting NAC resistance (FIG. 5D). In particular, high GLS1 expression was significantly correlated with tumor upregulation of GSR proteins (p=0.038), supporting the coordinated activation of pathways involved in GSH dynamics (FIG. 5E).

Overall, these results demonstrated that the expression of GSH metabolic proteins, especially GLS1, is clinically important in defining the molecular features of MIBC subtypes by NAC response.

2-6. Deep Learning-Based Analysis of Transcriptome Datasets from Multi-Cohort MIBC Patients

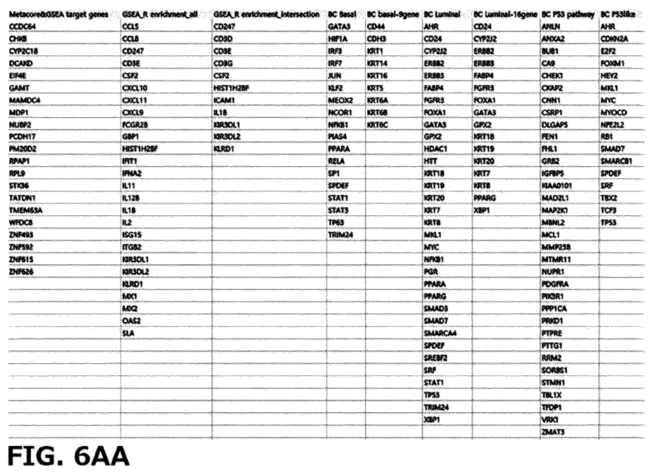

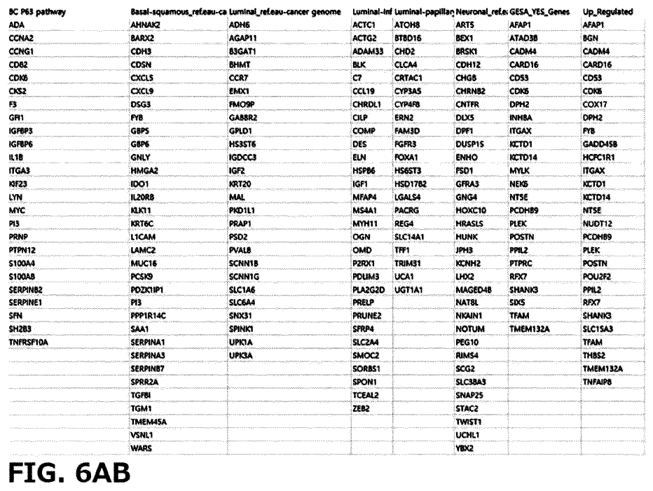

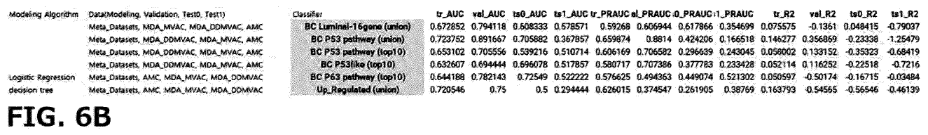

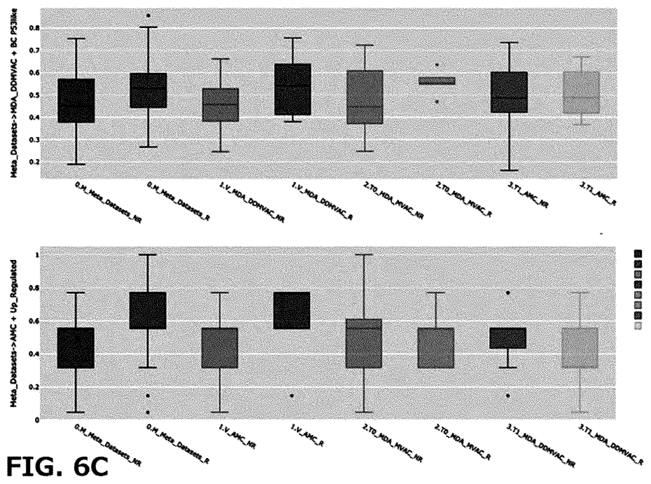

In order to discover biomarkers for predicting neoadjuvant chemotherapy (NAC) response that can be commonly applied to the multicohort datasets for muscle-invasive bladder cancer (MIBC) patient samples constructed in the above examples, 1) transcriptome datasets of our AMC validation cohort and three external cohorts (MDA_MVAC, MDA_DDMVAC, NAC_metadata) used in the present invention, 2) novel biomarker gene sets for predicting NAC response, such as GSH metabolism and immune response discovered in this study, and 15 molecular classifiers developed for MIBC molecular classification in a previous study (FIG. 6A) were all used to optimize the molecular classifiers that significantly affect the deep learning-based cross-validation analysis modeling and NAC responsiveness classification.

To compensate for the differences in datasets between our center and external cohorts, preliminary work was performed to select the optimal scaler and less than 10 optimal genes in each molecular classifier. First, 16,999 genes that existed in all four datasets were selected for cross-validation. Among the nine scaling algorithms (log2, log2_minmax, standard, minmax, max_abs, robust, power, quantile, rankgauss), the standard, power, and rankgauss scalers, which are scalers that follow the Gaussian distribution and can support the null hypothesis (the means of the two distributions are the same) with a p-value of 0.01 or higher in the pairwise T-test of the four patient cohort datasets, were selected. In order to optimize variables by random forest for each classifier and scaler, 1) when the number of genes (variables) in the classifier was 10 or less, it was maintained as is, 2) when the number of genes (variables) in the classifier exceeded 10, the top 10 main variables of the random forest model were selected, and 3) the sum of the performances of each random forest model, Accuracy, Recall, Precision, F1, MCC, and R2, was added to the performance score of the scaler. As a result, the power scaler was finally selected as the optimal scaler, and the top 10 gene sets within each of the 16 molecular classifiers were optimized.

For cross-validation modeling, one of the four patient cohort datasets standardized with power scaling determined in the preprocessing task was used for training, one for validation, and the remaining two for testing, performing 12 sets of cross-validation. At this time, a model was created using the first data from each dataset pair for each molecular classifier according to logistic regression, decision tree, and random tree statistical techniques, and validation and testing tasks were performed. The criteria selected for cross-validation were 1) the validity of the difference between NAC response (R) and non-response (NR) groups within the same dataset, 2) the significance between the R groups of each cohort, and 3) the significance between the NR groups of each cohort, and based on these criteria, a total of 612 (12×3×17=612) cross-validations were performed, and the same process as above was applied to the new AMC union classifier discovered by our research team, performing a total of 1,224 validation analyses.

As a result of performing deep learning cross-validation with this work process, by modeling Meta_Datasets (NAC_metadata), under the conditions validated in the MDA cohort, BC luminal-16 genes, BC p53 pathway, and BC p53-like pathway molecular classifiers could significantly distinguish between NAC NR and R groups in logistic regression modeling, and when our cohort (AMC validation cohort) was validated, BC p63 pathway was selected as a significant classifier and optimized in the logistic regression model, and in decision tree modeling, AMC Up-regulated molecular classifier constructed by our research team was confirmed to significantly distinguish between NAC NR and R groups (FIG. 6B). In particular, for the BC p53-like pathway, differences were observed between the NAC NR and R groups in the AMC cohort, and this was confirmed to be valid for all three other external cohorts (FIG. 6C).

### 2-7. Identification of Prospective Biomarkers to Predict NAC Response in MIBC Patients

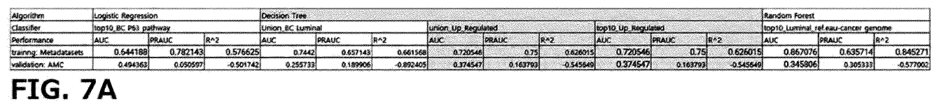

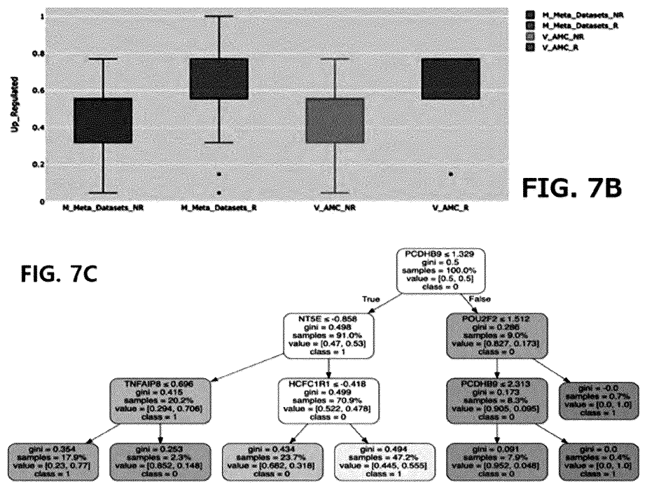

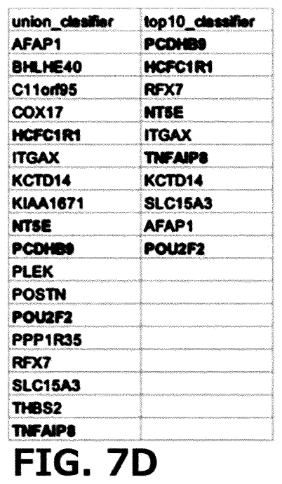

In this example, the clinical significance and validity were evaluated by utilizing the digital pathology analysis method constructed in the present invention among biomarkers in the optimized molecular classifier that has a significant function in predicting NAC response in the deep learning-based multi-cohort transcriptome analysis modeling analysis method. To this end, the NAC_metadata cohort, which showed the highest statistical significance in transcriptome analysis modeling, was selected and cross-validated using the same modeling method as the AMC validation cohort transcriptome datasets. As a result, in logistic regression modeling, the BC p63 pathway was selected as a molecular classifier that distinguishes NAC response, in random forest modeling, the Luminal_ref. EUA_cancer genome classifier was selected, and in decision tree modeling, it was confirmed that BC luminal and AMC Up-regulated molecular classifiers were molecular classifiers that could significantly distinguish NAC NR and R groups (FIG. 7A). In logistic regression modeling, the optimal molecular classifiers that distinguish between NAC NR and R groups included key genes such as ITGAX, AFAP1, KCTD14, RFX7, DPH2, SLC15A3, PPIL2, CARD16, FYB, GADD15B, and DNMT3L. In addition, for the decision tree modeling that showed the best significance (FIGS. 7B and 7C), the molecular classifiers included the key genes PCDHB9, HCFC1R1, NT5E, TNFAIP8, and POU2F2 (FIGS. 7C and 7D).

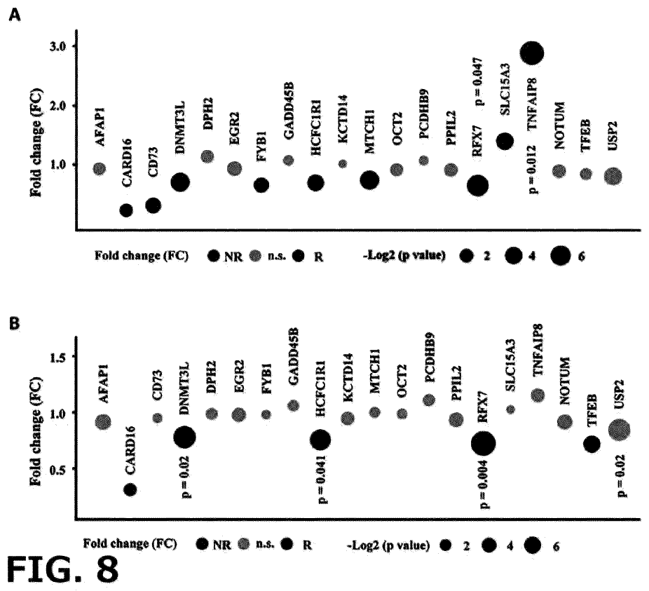

In order to validate the clinical significance of the key genes derived from the transcriptome-related deep learning modeling analysis, the difference in protein expression of the corresponding genes in bladder cancer samples from patients in the NAC NR and R groups was evaluated using a digital pathology analysis method that applied the tumor/stroma classifier developed in the present invention. As a result, in the AMC discovery cohort, the expression of CARD16, CD73, DNMT3L, FYB1, HCFC1R1, MTCH1, RFX7, SLCC15A3, and TNFAIP8 proteins showed expression differences in the two groups according to NAC responsiveness (FIG. 8A), and in particular, the expression differences of CARD16, DNMT3L, HCFC1R1, RFX7, and USP2 proteins showed significant differences between the NAC NR and R groups in clinical samples of patients in the AMC validation cohort (FIG. 8B). Therefore, we developed an analytical modeling technique that can predict NAC responsiveness and key genes that determine it through deep learning modeling of multi-cohort transcriptomes of clinical samples of muscle-invasive bladder cancer, and validated the clinical significance of these novel biomarkers at the protein change level through digital pathology analysis.

### 2-8. Deep Learning-Based Digital Pathology Analysis for Predicting NAC Response in MIBC Patients

In this study, a total of 61 genes were selected as biomarkers for predicting bladder cancer NAC responsiveness, including 1) GSH cell metabolism genesets, 2) immune response-related genesets, and 3) molecular classifiers optimized by deep learning-based transcriptome cross-validation modeling, and digital pathology analysis datasets for bladder cancer samples from 63 patients whose NAC responsiveness could be confirmed were secured at our center, and the 61 protein markers used for digital pathology analysis in the present invention are as follows: GLS1, PSAT1, CBS, GCLC, GCLM, Gluta (GSR), GlnRS (QARS1), GGT7, Perox (PRDX1), PLOD2, RPAP1, RPL9, MITF, CD44v6, CDK1, FZD9, GAD2, PPP2R5A, non-p (b-catenin), B-catenin, SALL4, SOX2, TFCP2L1, TFEB, ICAM1, TRAF2, TRAF6, IL15RA, AFAP1, CARD16, CD11c, CD73, CD3Z, EBI3 (IL27B), EMX1, DNMT3L, DPH2, EGR2, FYB1, GADD45B, HCFC1R1, KCTD14, MTCH1, OCT2, PCDHB9, PPIL2, RFX7, SLC15A3, TNFAIP8, ANPEP, BDN, EOGT, FOXA1, KIFC2, KIR3DL1, NOTUM, USP2, CCDC6, CK5 (KRT5), CK20 (KRT20), GATA3.

The ultimate goal of this research and development technology is to discover the optimal protein combination for immunohistochemical staining that can predict NAC responsiveness by utilizing digital pathology datasets of muscle-invasive bladder cancer (MIBC) patient samples. To utilize a digital pathology dataset with large patient clinical samples and a large number of antibodies, a deep learning analysis method similar to that used in the previous transcriptome cross-validation modeling study was applied. Because the immunostaining results of external cohorts were not available, for cross-validation of the digital pathology analysis results, an independent cohort (group 4) was established that included 84 patients (Non-cT2-4aNOM0) who received NAC treatment as a first line at our center, along with the NAC treatment patient group (cT2-4aNOM0) cohort used in the development of this study, and digital pathology analysis was performed on bladder cancer clinical samples from these cohorts using the same method.

The entire process of deep learning modeling of the digital pathology dataset is as follows. First, for the tumor compartment results, all values were present, but for the stroma compartment results, two patient observation values were lost and were therefore excluded from the analysis. A total of 10 gene pools (5 tumors, 5 stroma) were extracted according to the data-based key biomarker extraction process described further below. In order to find the optimal model, for each extracted gene pool, 1) the number of valid markers was repeated from 2 to 5, 2) the base model was repeated with decision tree and logistic regression, 3) the training data (NAC treatment patient group; group 1+2) and the first-line validation patient group (group 4) were combined and randomly performed 3-fold cross-validation to select the optimal markers corresponding to the number of valid markers, and 4) the variable selection process was performed in both forward and backward. The forward process used here is a method of sequentially adding one marker that can produce the optimal effect together with the already selected markers from the selectable marker pool until the number of valid markers is reached, and the backward process is a method of sequentially removing markers one at a time from the entire marker pool until the number of effective markers is reached, thereby achieving the optimal effect. By repeating this analysis method, if a new model satisfying the conditions presented in FIG. 9A is derived, it is selected as the optimal model. Unlike transcriptome cross-validation modeling, digital pathology modeling has limitations in that 1) the total number of measured biomarkers is small (61), 2) the number of patients in the training data (55 cases) and validation data (36 cases) is small, and 3) the clinical characteristics of the patient groups included in the training and validation data are different. Therefore, a data-based key biomarker extraction process was applied. To this end, we selected the recursive feature elimination with cross validation (RFECV) analysis method, which sequentially removes one variable from the dataset pool containing all variables and modeling them, and gives a higher rank to the variable if the performance is better than the previous model, and gives a lower rank to the variable if not (FIG. 9B). Cross-validation of the RFECV process allowed us to create a variable ranking with enhanced discriminatory power, and the modeling methods used in the iterative modeling process were decision tree (tree) and logistic regression (reg).

Data types using the digital pathology observation values themselves (raw) and standardized (std) datasets were created and used for analysis. Except for mean_tumor_vals, 2 to 5 optimal valid markers were extracted for each forward and backward of the Recursive Feature Selection method through decision tree and logistic regression modeling for each of the two data types, and then modeling was performed on the training data with the corresponding base model. In the case of setting mean_tumor_vals, the same work was performed on std data as well as raw data. For each tumor and stroma compartment result, data type, and modeling technique, protein groups that can distinguish between the two groups of NAC response NR and R were selected (FIGS. 9C and 9D), and among the two models finally derived, the optimal model was selected from the gene pool of tumor_tree_vals (raw data type, decision tree model) (FIG. 9E). The specific results and key genes of each decision tree model selected as the optimal model in the digital pathology dataset for the tumor and stroma compartments are shown in FIGS. 9F and 9G. Therefore, in the present invention, through high-speed automated digital pathology analysis of large-scale muscle-invasive bladder cancer clinical samples and deep learning-based modeling of the digital pathology results on the expression levels of a large number of proteins expressed in tumors and stroma, optimal protein combination groups (FIGS. 9C and 9D) capable of predicting anticancer treatment responsiveness were developed.

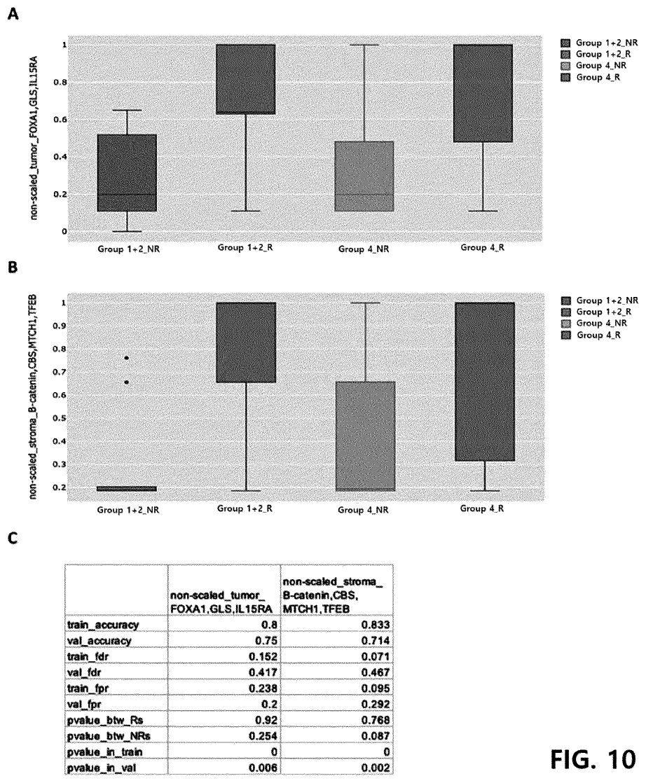

In order to validate the usability of the deep learning-based digital pathology modeling analysis method developed in this study, a cross-validation analysis was performed using an independent cohort consisting of first-line bladder cancer patients. As a result, it was confirmed that in the case of modeling based on tumor compartment data, there was a significant difference in the distribution of NAC responsiveness between both the training and validation patient groups (A of FIG. 10). However, in the case of modeling based on stroma compartment data, it was confirmed that the distinction for NAC response was not clear in the validation cohort consisting of the first-line patient group (B of FIG. 10), and it is judged that the reason for this is that the frequency of deriving highly significant variables in both training and validation data from variable selection is low.

However, the above examples are only intended to illustrate the content of the present invention, and the scope of the present invention is not limited to the above examples. Examples of the present invention are provided to describe the present invention more fully to those skilled in the art.

1. (canceled)

2. (canceled)

3. (canceled)

4. (canceled)

5. (canceled)

6. (canceled)

7. A composition for diagnosing chemotherapy resistance in a solid cancer patient, comprising an agent for measuring an expression level of a glutathione-related gene.

8. The composition of claim 7, wherein the glutathione-related gene is at least one selected from the group consisting of GLS1, PSAT1, CBS, GCLC, GCLM, Gluta (GSR), GlnRS (QARS1), GGT7, Perox (PRDX1), PLOD2, RPAP1, RPL9, MITF, CD44v6, CDK1, FZD9, GAD2, PPP2R5A, non-p (b-catenin), B-catenin, SALLA, SOX2, TFCP2L1, TFEB, ICAM1, TRAF2, TRAF6, IL15RA, AFAP1, CARD16, CD11c, CD73, CD3Z, EBI3 (IL27B), EMX1, DNMT3L, DPH2, EGR2, FYB1, GADD45B, HCFC1R1, KCTD14, MTCH1, OCT2, PCDHB9, PPIL2, RFX7, SLC15A3, TNFAIP8, ANPEP, BDN, EOGT, FOXA1, KIFC2, KIR3DL1, NOTUM, USP2, CCDC6, CK5 (KRT5), CK20 (KRT20), GATA3, BHLHE40, C11orf95, REX7, COX17, NTSE, ITGAX, KIAA1671, NT5E, POU2F2, PLEK, POSTN, PPP1R35, THBS2, and INFAIP8.

9. The composition of claim 7, wherein the agent for measuring the expression level of the glutathione-related gene comprises a peptide, an antibody, and a primer that specifically bind to the glutathione-related gene.

10. A kit for diagnosing chemotherapy resistance in a subject having a solid cancer, comprising the composition of claim 7.

11. A method for determining chemotherapy resistance in a subject having a solid cancer, the method comprising:
measuring an expression level of a glutathione-related gene from a sample isolated from the subject;
comparing the expression level of the glutathione-related gene with that of a normal control group sample; and
determining that the subject has a chemotherapy resistance when the expression level of the glutathione-related gene measured from the sample isolated from the subject is different from that of the normal control group sample.

12. The method of claim 11, wherein the expression level of the glutathione-related gene is measured by at least one method selected from the group consisting of Western blot, enzyme-linked immunosorbent assay (ELISA), immunohistochemical staining, immunoprecipitation, immunofluorescence, transcriptome, epigenome, and a quantitative real-time PCR technique.

13. The method of claim 11, wherein the glutathione-related gene is at least one selected from the group consisting of GLS1, PSAT1, CBS, GCLC, GCLM, Gluta (GSR), GlnRS (QARS1), GGT7, Perox (PRDX1), PLOD2, RPAP1, RPL9, MITF, CD44v6, CDK1, FZD9, GAD2, PPP2R5A, non-p (b-catenin), B-catenin, SALL4, SOX2, TFCP2L1, TFEB, ICAM1, TRAF2, TRAF6, IL15RA, AFAP1, CARD16, CD11c, CD73, CD3Z, EBI3 (IL27B), EMX1, DNMT3L, DPH2, EGR2, FYB1, GADD45B, HCFC1R1, KCTD14, MTCH1, OCT2, PCDHB9, PPIL2, RFX7, SLC15A3, TNFAIP8, ANPEP, BDN, EOGT, FOXA1, KIFC2, KIR3DL1, NOTUM, USP2, CCDC6, CK5 (KRT5), CK20 (KRT20), GATA3, BHLHE40, C11orf95, REX7, COX17, NTSE, ITGAX, KIAA1671, NT5E, POU2F2, PLEK, POSTN, PPP1R35, THBS2, and INFAIP8.

14. The method of claim 11, wherein the sample is at least one selected from the group consisting of saliva, urine, tissue, whole blood, serum, and plasma.

15. A method for treating a solid cancer in a subject in need thereof, the method comprising:
measuring an expression level of a glutathione-related gene from a sample isolated from the subject;
comparing the expression level of the glutathione-related gene with that of a normal control group sample;
determining that the subject has a chemotherapy resistance when the expression level of the glutathione-related gene measured from the sample isolated from the subject is different from that of the normal control group sample; and
administering to the subject an anticancer agent.

16. The method of claim 15, wherein when the subject is determined to not have a chemotherapy resistance, the anticancer agent is a neoadjuvant chemotherapy agent.

17. The method of claim 16, wherein the neoadjuvant chemotherapy agent is a platinum agent.

18. The method of claim 17, wherein the platinum agent is cisplatin.

19. The method of claim 15, wherein the glutathione-related gene is at least one selected from the group consisting of GLS1, PSAT1, CBS, GCLC, GCLM, Gluta (GSR), GlnRS (QARS1), GGT7, Perox (PRDX1), PLOD2, RPAP1, RPL9, MITF, CD44v6, CDK1, FZD9, GAD2, PPP2R5A, non-p (b-catenin), B-catenin, SALL4, SOX2, TFCP2L1, TFEB, ICAM1, TRAF2, TRAF6, IL15RA, AFAP1, CARD16, CD11c, CD73, CD3Z, EBI3 (IL27B), EMX1, DNMT3L, DPH2, EGR2, FYB1, GADD45B, HCFC1R1, KCTD14, MTCH1, OCT2, PCDHB9, PPIL2, RFX7, SLC15A3, TNFAIP8, ANPEP, BDN, EOGT, FOXA1, KIFC2, KIR3DL1, NOTUM, USP2, CCDC6, CK5 (KRT5), CK20 (KRT20), GATA3, BHLHE40, C11orf95, REX7, COX17, NTSE, ITGAX, KIAA1671, NTSE, POU2F2, PLEK, POSTN, PPP1R35, THBS2, and INFAIP8.

20. The method of claim 15, wherein the sample is at least one selected from the group consisting of saliva, urine, tissue, whole blood, serum, and plasma.

21. The method of claim 15, wherein the expression level of the glutathione-related gene is measured by at least one method selected from the group consisting of Western blot, enzyme-linked immunosorbent assay (ELISA), immunohistochemical staining, immunoprecipitation, immunofluorescence, transcriptome, epigenome, and a quantitative real-time PCR technique.

22. The method of claim 15, wherein the solid cancer is muscle-invasive bladder cancer.
